SEQUENCE LISTING (17 sequences)

SEQ ID NO: 1 - DNA, 5139 bp, Lactuca serriola
  misc_feature 1..5139: note = cDNA
  source 1..5139: mol_type = other DNA
atgtcttcccacaaacagtttgatcacttacgaatcccgcttgaagagatagtatctgccaccgacaatttttctgatgcaaatttcatcagacatgatggatttggaaaggagtacaaaggacaactcttgcagtccggtcgattggtcgatatcgttgcacgaaggttagatgataagaatggaataggaatcaaagagttttggatggaggtctcgatgctttgtacacttgagcatcaaaatatcgtctctatcgttgggttttgtgacgatggtgatgatggcgagaagatcgtcataaacaagcgtgagagtacccgtggaagtgtttgtaactatctaagagacccgagcttcacatggatgcgaaggttgcggatatgtgttggtgttgcgcgggcattaaattacattcattatgatgagggacgtgattttggcgtcattcatcgcgatatcaagagttcaaagattttattagatgacaactgggaagcaaaattatctggttttgaactttccatgaatcaggcagcagctgaaaggaactgtccttgccagctatctggattttgtggcacagtgggatacatggacccgacatattttaagactggaagtgtgtcccacaagtccgatatgttttcctttggcgtggtattaattgaagtactttgtggcaaggtaacagaagctgtaaacgagggtgaggactatctatatgaattggccaaatcacatattgaaaatggaagactatatgatatggtgaatccgggtctatggaaacaaatggacccacagtcattcaaaatcttttcagaaatagcatattgttgcttgaaggcagaaccatcagaacgaccaagcatagatcagattctcatacaacttgagaaagcactggaatctcaacaggaacaagaaaatcatcaagaaaagattgaaggcgcaatatccaagagcttgaagttggctcacttacaaatcccacttgaagagatagtatcagccaccaacaacttttctgatgcaaaccttatcggacgtgatgggtttggaaaggagtacaaaggacaactcttggggtcacatgaattagtcaatattgttgcgcggaggtattttgatgataactcaaggagagaaagcaacgtgttttttatggaggtttcgatgctttctaaacttaagcataaaaatcttgtctctatcattgggttttgtgatgagaacggtgagaagatcgtcataaacaagcatgagagccgtggaaatctgtatgactatctacgagacccgaccctcacttggatgcgaagattgcagatatgtgttggggccgcacacgctttgagttacatttattatgataagcgacgtgatttcagcatcatacaccgtgatatcaagagctccaagattttactggatgagcagtgggaagcaaaattatctggttttgaactttccatcactcagacagcaagtcgaagggaccgtctttgcgttcctgcggatgtttgtggtacgatagggtaccgtgacccaacatatgttcagagtggaatcgtgacccacaagtcagatatttattcatttggcattgtattatttgaagtcctgtgcggaaagcattgtgatgatgataagttgttagctccatcagccagatcatactatgaagagggaagactagaagatatgatagatccggttctatggaaacaaatggacccaaaatcattcaaaatcttttcggaaacagcatactactgcgtgaaggaacaacgacaacaacgtccaaatatacatcagattgtcatacaacttgagaaagccttggaatttcaatggaaacatcaaaaccctgatttcatatcaaaggctcactctatggttaagggcatgacttccaagagcttgaaggggaaaaacttgaaacacttagagatcccactcattgatatacaattggccaccgagaattttggtaaaacataccccattgggtcaggcggatatggtgatgtgtacaaagctgaacttgaccattttgatagcaaaaacttgttgccattagaagagaagaagacaagggaacatgccaagaagcgctccactgtagctatcaaacgcatattgaatagagaagacaaacaaggaatacaagggttcctagcagaaattgaaatgcttacaaattgtaagcatcccaacataatctctcttcttggattttgcaaggaaggtgatcatatgttacttgtctacgagcatgcttctaaaggaagccttagtgattatttgggaagcaccggtggtaagttgactaatcttagttgggtgcaacgtataaagatatgtattgatatcgcacgcggattgaattaccttcataccaagatagaggacgatcgaaggataatacaccgtgatataaaaagcgataacattttgttaactgagaattggcaagccaagattgctgattttgggctctccagattacgcagtgtgaaaaaaagtcaacaagtgaacaagactctcaatacagaaactcgtgcaggcacagatttttattgggacccagaatattttaaaacgggtaggttgaaaaatgaatcagacatttactcttttggggtcgttttatttgagatcctgtcagggaagcttgccaatgatgaaatttacaccaaagaaaatatcaacgggattgcacctgtcgcaagacgtcgcttcaatgagggaacaataagggaaatggtagatcctaagttgatggaagaaactcatgacaacattttcacttcaagtagaggacccgacaaagattcattggatacatttacaaaaattgcataccaatgtgttgctgaatctcaaactaaccgtccaacagcagaagtcataattaaggaacttgaggaagcgttatctttacaagaaaacatcaaggaccacctccaattctcacttgatgacattaaaactgccacacaagacttcagtcaaaacaacttgattggtagtggagcatttgggaatgtatataaaggagaagttatgcatgctaacggacgtaatgccattgctgcaaagcggtgggatgcgagtggtgatcagggagaacaccaatttttgatggagcttcaaattctcttggaatataaacatgaaaattgtattggtcttttaggctattgcaacgaaatgggtgaaaaaatcattgtttatgagtatgcttctagaggaagtctggataggtacttgaaggacaaaggtcttatgtggatgaaacggcttgagatatgcatcgatattgcaagtgggttggatttccttcatggaggtggagcggcacaagagccggtgatgcatagagacattaaaagcgcaaacgttctattaaccaatgattggaaaggaaaaatttctgattttggggtttcgttaataagtccattaaataaggatattgactttgtctttgataatgcttgcggcacaagaggttatgttgacccactatacctgaaattgggtttcttaaccaaagaatctgatatatattcactcggcgttgttttatttgagattttgtctggcagagatgcatatgttgtttacaatacgcatgggcatctatctgtatgggttaaaaacaacttcaatgagggaaaacttgatgagatggtgtttgaaggtataaaagaacaaattgccccaaaatcgttggctacatttcaaatgattgcctaccaatgcttacatgatgataataaaaaacgaccaacatcacgtgatgtgctactacaactcaagaaatcgttggaattccaagaggactatgaaatatgggagcgcaaattgcccgatgattatgaacaaatatttaaaatctcaaatttttcggagatctactccactaaaaagaaaaaggacctccacaacttgctctccacaggaatcatccttcaaaaaggcacagagtggttttcaataggtgctaatggagaaagaaatgaaatgatatcagcatggaagtttttatacaaacaacgtcagtcacataagtggggatatgttccacaatcaaggtttcaaaaggtagcagagatgctagatttttcgaatctaaagatcaaaatgaagataaaaactcaattattatcttcgggcgtcaattatgggattcatctcatctttagaatttgtggtccaagaaaatctctagctaagcagatgtacgtaaaccttaagtacaaaaaggggagagggagcgaaaccttacatgcatatttcgcaacatggagagaagatgattggatgatgattgaactgtgtcgattctcaaatgacaaggaaaatattgacttggagtttctgctagaaagcctttcacgatactattgtggaagccatggcatatatgttgaaggcattgagtttcgagccattgataatgtgaaacatgaagaaacggatgaaatacaagaagtgcaacaagtcctgaaatcagactcaaacatgaatctcatgcaacaattgccaactaaccttgaagaaatatttaagagatctgaaaatgaggatggagatgaaaagctgtttttgctcaacgaggtgaacggaaagagatatcttatgctttcagcaaaggcggctctttacaactattctgatgtgaagcttttcaaaacaaaaccatcaattcaatccagatttctagaggtgattgagcttctatcacaacaagtacttcgtatcaattgcaagattaaaagtcaaatgttatcacgagatacagactacacgtgttatcttgtgttcaagctttcagaaaagtgtcgtgggctgcattgtcctgtgaaagtacgaaatctacttcaccggaacaagaaagaggttggaattatttattttagaaccccaaatccatggaatttacatgatactaatcaagctccacaacaaaggccagatggatggatggaagttaaggtctggaaattcaacctaaatcatgaactcataaaaaatgatagttttcgtgtgaatttgaagttaattagttatgaagggactctatccggccttattttatgtggccttgaatttcggccactgtaa

SEQ ID NO: 2 - protein, 1712 aa, Lactuca serriola
  REGION 1..1712: note = protein
MSSHKQFDHLRIPLEEIVSATDNFSDANFIRHDGFGKEYKGQLLQSGRLVDIVARRLDDKNGIGIKEFWMEVSMLCTLEHQNIVSIVGFCDDGDDGEKIVINKRESTRGSVCNYLRDPSFTWMRRLRICVGVARALNYIHYDEGRDFGVIHRDIKSSKILLDDNWEAKLSGFELSMNQAAAERNCPCQLSGFCGTVGYMDPTYFKTGSVSHKSDMFSFGVVLIEVLCGKVTEAVNEGEDYLYELAKSHIENGRLYDMVNPGLWKQMDPQSFKIFSEIAYCCLKAEPSERPSIDQILIQLEKALESQQEQENHQEKIEGAISKSLKLAHLQIPLEEIVSATNNFSDANLIGRDGFGKEYKGQLLGSHELVNIVARRYFDDNSRRESNVFFMEVSMLSKLKHKNLVSIIGFCDENGEKIVINKHESRGNLYDYLRDPTLTWMRRLQICVGAAHALSYIYYDKRRDFSIIHRDIKSSKILLDEQWEAKLSGFELSITQTASRRDRLCVPADVCGTIGYRDPTYVQSGIVTHKSDIYSFGIVLFEVLCGKHCDDDKLLAPSARSYYEEGRLEDMIDPVLWKQMDPKSFKIFSETAYYCVKEQRQQRPNIHQIVIQLEKALEFQWKHQNPDFISKAHSMVKGMTSKSLKGKNLKHLEIPLIDIQLATENFGKTYPIGSGGYGDVYKAELDHFDSKNLLPLEEKKTREHAKKRSTVAIKRILNREDKQGIQGFLAEIEMLTNCKHPNIISLLGFCKEGDHMLLVYEHASKGSLSDYLGSTGGKLTNLSWVQRIKICIDIARGLNYLHTKIEDDRRIIHRDIKSDNILLTENWQAKIADFGLSRLRSVKKSQQVNKTLNTETRAGTDFYWDPEYFKTGRLKNESDIYSFGVVLFEILSGKLANDEIYTKENINGIAPVARRRFNEGTIREMVDPKLMEETHDNIFTSSRGPDKDSLDTFTKIAYQCVAESQTNRPTAEVIIKELEEALSLQENIKDHLQFSLDDIKTATQDFSQNNLIGSGAFGNVYKGEVMHANGRNAIAAKRWDASGDQGEHQFLMELQILLEYKHENCIGLLGYCNEMGEKIIVYEYASRGSLDRYLKDKGLMWMKRLEICIDIASGLDFLHGGGAAQEPVMHRDIKSANVLLTNDWKGKISDFGVSLISPLNKDIDFVFDNACGTRGYVDPLYLKLGFLTKESDIYSLGVVLFEILSGRDAYVVYNTHGHLSVWVKNNFNEGKLDEMVFEGIKEQIAPKSLATFQMIAYQCLHDDNKKRPTSRDVLLQLKKSLEFQEDYEIWERKLPDDYEQIFKISNFSEIYSTKKKKDLHNLLSTGIILQKGTEWFSIGANGERNEMISAWKFLYKQRQSHKWGYVPQSRFQKVAEMLDFSNLKIKMKIKTQLLSSGVNYGIHLIFRICGPRKSLAKQMYVNLKYKKGRGSETLHAYFATWREDDWMMIELCRFSNDKENIDLEFLLESLSRYYCGSHGIYVEGIEFRAIDNVKHEETDEIQEVQQVLKSDSNMNLMQQLPTNLEEIFKRSENEDGDEKLFLLNEVNGKRYLMLSAKAALYNYSDVKLFKTKPSIQSRFLEVIELLSQQVLRINCKIKSQMLSRDTDYTCYLVFKLSEKCRGLHCPVKVRNLLHRNKKEVGIIYFRTPNPWNLHDTNQAPQQRPDGWMEVKVWKFNLNHELIKNDSFRVNLKLISYEGTLSGLILCGLEFRPL

SEQ ID NO: 3 - DNA, 5133 bp, Lactuca saligna
  misc_feature 1..5133: note = cDNA
  source 1..5133: mol_type = other DNA
atgtctccccacaaacagtttgatcacttacgaatcccgcttgaagagatagtatctgccaccgacaacttttctgatgcaaatttcatcagatatgatggatttggaaaggagtacaaaggacaactcttgcagtccggtcgattggtcgatatcgttgcacggaggttagatgataagaatggaataggaatcaaagagttttggatggaggtctcgatgctttgtacacttgagcatcaaaatatcgtctctatcgttgggttttgtgacgatggtgatgatggtgagaagatcgtcataaacaagcttgagacccgtggaagtctttgtaactatttaacagacccgtgcctcacatggatgcgaaggttgcagatatgtgtcggtgttgcgcgggcattatattacattcattatgatgagggacgtgattttggcgtcattcatcgcgatatcaagagttcaaagattttactagatgacaactgggaagcaaaattatatggttttgaactttccatgaatcaggctgcggctgaatggaactgtgtatcttataataaagtttgtggcacacccggatatatggacccaacatatgtcaagactggaagtgtgtcccacaagtccgatatgttttcctttggcgtggtattaattgaagtactttgtggcaaggtaacagaagctgtaaacgagggtgaggactatctatatgaattggccaaatcacatattgaaaatggaagactatatgatatggtgaatccgggtctatggaaacaaatggacccacaatcattcaaaatcttttcagaaatagcgtattgttgcttgaaggcagaaccatcagaacgaccaagtatagatcagattctcatacaacttgagaaagcactggaatctcaacaggaacaagaaaatcatcaagaaaagattgaaggcgcaacatccaagagcttgaagttggctcacttacaaattccacttgaagagatagtatcagccaccaacagcttttctgatgcaaaccttatcggacgtgatgggtttggaaaggagtacaaaggacaactcttgcggtcacatgaattagtcaatattgttgcgcggaggtttttttatgataacttaaggagagaaagcaacgggttttttatggaggtttcgatgctttctaaacttaagcatagaaatcttgtctctatcattgggttttgtgatgagaacggtgagaagatcgtcataaacaaacatgagatccatggaagtctgtacgactatctacaagacccgaccgtgacatggatgcgaagattgcagatatgtattggtgccgcgcacgctttgagttacatttattatgataagcgacgtgatttcagcatcatacaccgtgatatcaagagctccaagattttactggatgagcagtgggaagcaaaattatctggttttgaactttccatcactcagacagcaagtcgaagggactgtctttgcattcctgtggatgtttgtggtacgatagggtaccgtgacccaacatatgttcagagtggattcgtgacccacaagtcagatatttattcatttggcattgtattatttgaagtcctgtgcggaaagcattgtgatgatgataagttgttagctccatcggccagatcatactatgaagagggaagactagaagatatgatagatccggttctatggaaacaaatggacccaaaatcattcaaaatcttttcagaaacagcatactactgcgtgaaggaacaacgacaacaacgtccaaatatacatcagattgtcatacaacttgagaaagccttggaatttcaatggaaacatcaaaaccctgctttcatatcaaaggctcactctatggttaagggcatgacttccaagagcttgaaggggaaaaacttgaaacacttagagatcccactcattgatatacaattggccaccgagaattttggtaaaacatacctcattgggtcaggcggatatggtgatgtgtacaaagctgaacttgaccattttgatagcaaaaacttgttgccattagaagagaagaagacaagggaacatgccaagaagcgctccactgtagctatcaaacgcatattgaatagagaagacaaacaaggaatacaagggttcctagcagaaattgaaatgcttacaaattgtaagcatcccaacataatctctcttcttggattttgcaaggaaggtgatcatatgttacttgtctacgagcatgcttctaaaggaagccttagtgattatttgggaagcaccggtggtaagttgactaatcttacttgggtgcaacgtataaagatatgtattgatattgcacgcggattgaattaccttcataccaagatagaggacgatcgaaggataatacaccgtgatataaaaagcgataacattttgttaactgagaattggcaagccaagattgctgattttgggctctccagattacgcagtgtgaaaaaaagtcaacaagtgaacaagactctcaatacagaaactcttgcaggcacagatttttattgggacccagaatattttaaaacgggtaggttgaaaaatgaatcagacatttactcttttggagtcgttttatttgagatcctgtcagggaagcttgctaatgatgaaatttacaccaaagaaaatatcaacgggattgcacctgtcgcaagacgtcgcttcaatgagggaacaataagggaaatggtagatcctaagctgatggaagaaactcatgacaacattttcacttcaagtagaggacccgacaaagattcattggatacatttacgaaaattgcatatcaatgtgtggcagaatctcaaactaaccgtccaacagcagaagtcataattaagaaacttgaggaagcgttatctttacaagaaaacatcaaggaccacctcaaattctcacttgatgacattaaaactgccacacaagacttcagccataacaacttgattggtcatggaggatttggggatgtatataaaggagaagttacacatgctaagggacgttctactattgctgcaaagcggtggcatgggagatatgatcagggagaacacgaatttttgatggagcttcaaattctcttggagtataaacatgaaaattgtattggtcttttaggctattgcaacgaaatgggtgaaaaaatcattgtttatgagtatgcttctagaggaagtctggataggtacttgaaggacaaaggtcttatgtggatgaaacggcttgagatatgcatcgatattgcaagtgggttggatttccttcatggaggtggagcggcacaagagccggtgatacatagagacattaaaagctcaaacgttctattaaccaatgattggaaaggaaaaatttctgattttgggctttcgttaataagtccattaaataaggatattgactttgtcattgataatttacgcggcacaataggctatattgacccactatacctgaaattgggtttcttaaccaaagaatctgatatatattcactcggcgttgttttatttgagatattgtctggcagagatgcatttgttgattacaatacgcatgggcatctatctgtattggttaaaaacaacttcaatgaaggaaaacttgatgagatggtgttcgaaggtataaaagaacaaattgccccaaaatcattggctacatttcaaataattgcctaccaatgcttacatgatgataataaaaaacgaccaacatcacgcgatgtgctactacaactcaaaaagtcgttggaattccaagaggactacgaaatatgggagcgcaaattgcccaatgattatgaacaaatatttaaaatgtcaaaattttcggagatctactctactgaaaagaaaaaagacctccacaacttgctctccaaaggaatcatccttcaaaaaggcacagagtggtttttagtaggtgctaatggaaaaagaaatcaaatgatatcagcatggaagtttttatacaaacaacgccagtcatataagtggggatatgttccatactcaaggtttcaaaaggtagcagagatgctagatttttcgaatctaaagatcaaaatgaagataaaaactcaattattatcttcgggcgtcaattatgggattcatctcatctttagaatttgtggtccaagaaaatctctagctaagcagatgtacgtaaaccttaagtacaaaaaggggagagggagcgaaaccttacatgcatatttcgcaacatggagagaagatgattggatgatgattgaactgtgtcgattctcaaatgacaaggaaaatattgacttggagtttctgctagaaagcctttcgcaatactattgtggaagccatggcatatatgttgaaggcattgagtttcgagccattgataatgtgaaacatgaagaaacggatgaaatacaagaagtgcaacaagtactgaaatcagactcaaacatgaatctcatgcaacaattgccaactaaccttgaagaaatatttaagagatctgaaaatgaggatggagatgaaaagctgtttttgctcaacaaagtgaacggaaagaaatatcttatgctttctgcaaaggctgctgtttacaactattctgatgtgaagcttttcaaaacaaaaccatcaattcaatccagatttctagaggtgattgagcttctaccacaacaagtacttcgtatcaattgcaagattaaaagtcaaatgttatcacgagatacagactacacatgttatcttgtgttcaagctttcagaaaagtgtcgtgggctgcattgtcctgtgaaagtaagaaatctacttcatcggaacaagaaagaggttgaaattatttattttagaaccccaaatacatggaatttacatgatactaatcaagctccacaacaaaggccagatggatggatggaagttaaggtctggaaattcaacctaaatcatgaactcataaaaaatgatagttttcgtgtgaatttgaagttaattagttatgaagggactctatccggccttattttatgtggccttgaatttcggccactgtaa

SEQ ID NO: 4 - protein, 1710 aa, Lactuca saligna
  REGION 1..1710: note = protein
MSPHKQFDHLRIPLEEIVSATDNFSDANFIRYDGFGKEYKGQLLQSGRLVDIVARRLDDKNGIGIKEFWMEVSMLCTLEHQNIVSIVGFCDDGDDGEKIVINKLETRGSLCNYLTDPCLTWMRRLQICVGVARALYYIHYDEGRDFGVIHRDIKSSKILLDDNWEAKLYGFELSMNQAAAEWNCVSYNKVCGTPGYMDPTYVKTGSVSHKSDMFSFGVVLIEVLCGKVTEAVNEGEDYLYELAKSHIENGRLYDMVNPGLWKQMDPQSFKIFSEIAYCCLKAEPSERPSIDQILIQLEKALESQQEQENHQEKIEGATSKSLKLAHLQIPLEEIVSATNSFSDANLIGRDGFGKEYKGQLLRSHELVNIVARRFFYDNLRRESNGFFMEVSMLSKLKHRNLVSIIGFCDENGEKIVINKHEIHGSLYDYLQDPTVTWMRRLQICIGAAHALSYIYYDKRRDFSIIHRDIKSSKILLDEQWEAKLSGFELSITQTASRRDCLCIPVDVCGTIGYRDPTYVQSGFVTHKSDIYSFGIVLFEVLCGKHCDDDKLLAPSARSYYEEGRLEDMIDPVLWKQMDPKSFKIFSETAYYCVKEQRQQRPNIHQIVIQLEKALEFQWKHQNPAFISKAHSMVKGMTSKSLKGKNLKHLEIPLIDIQLATENFGKTYLIGSGGYGDVYKAELDHFDSKNLLPLEEKKTREHAKKRSTVAIKRILNREDKQGIQGFLAEIEMLTNCKHPNIISLLGFCKEGDHMLLVYEHASKGSLSDYLGSTGGKLTNLTWVQRIKICIDIARGLNYLHTKIEDDRRIIHRDIKSDNILLTENWQAKIADFGLSRLRSVKKSQQVNKTLNTETLAGTDFYWDPEYFKTGRLKNESDIYSFGVVLFEILSGKLANDEIYTKENINGIAPVARRRFNEGTIREMVDPKLMEETHDNIFTSSRGPDKDSLDTFTKIAYQCVAESQTNRPTAEVIIKKLEEALSLQENIKDHLKFSLDDIKTATQDFSHNNLIGHGGFGDVYKGEVTHAKGRSTIAAKRWHGRYDQGEHEFLMELQILLEYKHENCIGLLGYCNEMGEKIIVYEYASRGSLDRYLKDKGLMWMKRLEICIDIASGLDFLHGGGAAQEPVIHRDIKSSNVLLTNDWKGKISDFGLSLISPLNKDIDFVIDNLRGTIGYIDPLYLKLGFLTKESDIYSLGVVLFEILSGRDAFVDYNTHGHLSVLVKNNFNEGKLDEMVFEGIKEQIAPKSLATFQIIAYQCLHDDNKKRPTSRDVLLQLKKSLEFQEDYEIWERKLPNDYEQIFKMSKFSEIYSTEKKKDLHNLLSKGIILQKGTEWFLVGANGKRNQMISAWKFLYKQRQSYKWGYVPYSRFQKVAEMLDFSNLKIKMKIKTQLLSSGVNYGIHLIFRICGPRKSLAKQMYVNLKYKKGRGSETLHAYFATWREDDWMMIELCRFSNDKENIDLEFLLESLSQYYCGSHGIYVEGIEFRAIDNVKHEETDEIQEVQQVLKSDSNMNLMQQLPTNLEEIFKRSENEDGDEKLFLLNKVNGKKYLMLSAKAAVYNYSDVKLFKTKPSIQSRFLEVIELLPQQVLRINCKIKSQMLSRDTDYTCYLVFKLSEKCRGLHCPVKVRNLLHRNKKEVEIIYFRTPNTWNLHDTNQAPQQRPDGWMEVKVWKFNLNHELIKNDSFRVNLKLISYEGTLSGLILCGLEFRPL

SEQ ID NO: 5 - DNA, 5136 bp, Lactuca serriola
  misc_feature 1..5136: note = cDNA
  source 1..5136: mol_type = other DNA
atgtctccccacaaacagtttgatcacttacgaatcccgcttaaagagatagtatctgccaccgacaacttttctgatgcaaatttcatcagatatgatggatttggaaaggagtacaaaggacaactcttgcagtccggtcgattggtcgatatcgttgcacggaggttagatgataagaatggaataggaatcaaagagttttggatggaggtctcgatgctttgtacacttgagcatcaaaatatcgtctctatcgttgggttttgtgacgatggtgatgatggtgagaagatcgtcataaacaagcttgagacccgtggaagtcttcgtaactatctaagagacccgagcctcacatggatgcgaaggttgcagatatgtgttggtgttgcgcgggcattaaattacattcattatgatgagggacgtgattttagcgtcattcatcgcgatatcaagagttcaaagattttactagatgacaactgggaagcaaaattatatggttttgaactttccatgaatcaggcagcagctgaaaggaactgtccttgccagctatctagattttatggcacagagggatacatggacccgacatattttaagactggaagtgtgtcccacaagtccgatatgttttcctttggcgtggtattaattgaagtactttgtggcaaggtaacagaagctgtaaacgagggtgaggactatctatatgaattggccaaatcacatattgaaaatggaagactatatgatatggtgaatccgggtctatggaaacaaatggacccacaatcattcaaaatcttttcagaaatagcgtattgttgcttgaaggcagaaccatcagaacgaccaagtatagatcagattctcatacaacttgagaaagcactggaatctcaacaggaacaagaaaatcatcaagaaaagattgaaggcgcaacatccaagagcttgaagttagctcacttacaaatcccacttgaagagatagtattagccaccaacaacttttctgatgcaaaccttatcggacgtgatgggtttggaaaggagtacaaaggacaactcttgcggtcacatcaatcagtcaatattgttgcacggaggtttttaagtgataactcaaggagagaaagcaaagagttttttatggaggtttcgatgctttctaaacttaagcataaaaatcttgtctctatcattgggttttgtgatgagaacggtgagaagatcgtcataaacaaacatgagatccatggaagtctgtaccactatctacaagacccgaccgtgacatggatgcgaagattgcagatatgtgttggtgccgcgcacgctttgagttacatttattatgataagcgacgtgatttcagcatcatacaccgtaatatcaagagctccaagattttactggatgagcagtgggaagcaaaattatctggttttgaactttccatcactcagacagcaagtcgaagggaccgtctttgcatccctgcggatgtttgtggtacgatagggtactgcgacccaacatatattcatagtggaatcgtgacccacaagtcagatatttattcatttggcattgtattacttgaagtcatgtgtggaaagccttcctatgatgataagttgttagctccatcggccagatccttctatgaagagggaagactagaagatatgatagatccggttctatggaaacaaatggacccaaaatcattcaaaatcttttcggaaacagcatactactgcgtgaaggaacaacgacaacaacgtccaaatatacatcagattgtcatacaacttgagaaagccttagaatttcaatggaaacatcaaaaccctgatttcatatcaaaggctcactctatggttaagggcatgacttccaagagcttgaaggggaaaaacttgaaacacttagagatcccactcattgatatacaattggccaccgagaatttcggtaaaacatacctcattgggtcaggcggatatggtgaggtgtacaaagcagaacttgaccattttgatagcaaaaacttgttgccattagaagagaagaagacaagggaacataccaagaagcgcgccactgtagctataaaacgcatattgaatagagaagacaaacaaggaatacaagggttcctagcagaaattgaaatgcttacaaattgtaagcatcccaacataatctctcttcttggattttgcaaggaaggtgatcatatgatacttgtctacgagcatgcttctaaaggaagccttagtgattatttgggaagcatcggtggtaagttgactaatcttacttgggtgcaacgtataaagatatgtattgatattgcacgcggattgaattaccttcataccaagatagaggatgatcgaaggataatacaccgtgatataaaaagcgataacattttgttaactgagaattggcaagccaagattgctgattttgggctctccagattacgcagtgtgaaaaaaagtcaacaagcgaacaagactctcaatacagaaactcttgcaggcacagatttttattgggacccagaatattttaaaacgggtaggttgaaaaatgaatcagacatttactcttttggggtcgttttatttgagatcctgtcagggaagcttgccaatgatgaaatttacaccaaagaaaatatcaacgggattgcacctgtcgcaagacgtcgcttcaatgagggaacaataagggaaatggtagatcctaagttgatggaagaaactcatgacaacattttcacttcaagtagaggacccgacaaagattcattggatacatttacaaaaattgcataccaatgtgttgctgaatctcaaactaaccgtccaacagcagaagtcataattaaggaacttgaggaagcgttatctttacaagaaaacatcaaggaccacctcaaattctcacttgatgacattaaaactgccacacaagacttcagccataacaacttgattggtcatggaggatttgggaatgtatataaaggagaagttacacatgctaagggacgttctactattgctgcaaagcggtggcatggggaatatgaacagggagaacacgaatttttgatggagcttcaaattctcttggaatataaacatgaaaattgtattggtcttttaggctattgcaacgaaatgggtgaaaaaatcattgcttatgagtatgcttctagaggaagtctggataggtacttgaaggacaaaggtcttatgtggatgaaacggcttgagatatgcatcgatattgcaagcgggttggatttccttcatggaggtggagtagcacaagagccggtgatgcatagagacattaaaagctcaaacgttctattaaccaatgattggaaaggaaaaatttctgattttgggctttcgttaataagtccattaaataaggatattgactttgtcattgataatttatgcggcacaataggctatattgacccactatacctgaaattgggtttcttaaccaaagaatctgatatatattcactcggcgttgttttatttgagatattgtctggcagagatgcatatgttgattacaatacgcatgggcatctatctgtattggttaaaaacaacttcaatgaaggaaaacttgatgagatggtgttcgaaggtataaaagaacaaattgccccaaaatcattggctacatttcaaataattgcctaccaatgcttacatgatgataataaaaaacgaccaacatcacgcgatgtgctactacaactcaaaaagtcgttggaattccaagaggactacgaaatatgggagcgcaaattgcccaatgattatgaacaaatatttaaaatgtcaaaattttcggagatctactctactgaaaagaaaaaagacctccacaacttgctctccaaaggaatcatccttcaaaaaggcacagagtggtttttagtaggtgctaatggaaaaagaaatcaaatgatatcagcatggaagtttttatacaaacaacgccagtcatataagtggggatatgttccatactcaaggtttcaaaaggtagcagagatgctagatttttcgaatctaaagatcaaaatgaagataaaaactcaattattatcttcgggcgtcaattatgggattcatctcatctttagaatttgtggtccaagaaaatctctagctaagcagatgtacgtaaaccttaagtacaaaaaggggagagggagcgaaaccttacatgcatatttcgcaacatggagagaagatgattggatgatgattgaactgtgtcgattctcaaatgacaaggaaaatattgacttggagtttctgctagaaagcctttcgcgatactattgtggaagccatggcatatatgttgaaggcattgagtttcgagccattgataatgtgaaacatgaagaaacggatgaaatacaagaagtgcaacaagtcctgaaatcagactcaaacatgaatctcatgcaacaattgccaactaaccttgaagaaatatttaagagatctgaaaatgaggatggagatgaaaagctgtttttgctcaacgaagtgaacggaaagaaatatcttatgctttctgcaaaggctgctgtttacaactattctgatgtgaagcttttcaaaacaaaaccatcaattcaatccagatttctagaggtgattgagcttctaccacaacaagtacttcgtatcaattgcaagattaaaagtcaaatgttatcacgagatacagactacacatgttatcttgtgttcaagctttcagaaaagtgtcgtgggctgcattgtcctgtgaaagtaagaaatctacttcatcggaacaagaaagaggttgaaattatttattttagaaccccaaatgcatggaatttacatgatactaatcaagctccacaacaaaggccagatggatggatggaagttaaggtctggaaattcaacctaaatcatgaactcataaaaaatgatagttttcgtgtgaatttgaagttaattagttatgaagggactctatccggccttattttatgtggccttgaatttcggccactgtaa

SEQ ID NO: 6 - protein, 1711 aa, Lactuca serriola
  REGION 1..1711: note = protein
MSPHKQFDHLRIPLKEIVSATDNFSDANFIRYDGFGKEYKGQLLQSGRLVDIVARRLDDKNGIGIKEFWMEVSMLCTLEHQNIVSIVGFCDDGDDGEKIVINKLETRGSLRNYLRDPSLTWMRRLQICVGVARALNYIHYDEGRDFSVIHRDIKSSKILLDDNWEAKLYGFELSMNQAAAERNCPCQLSRFYGTEGYMDPTYFKTGSVSHKSDMFSFGVVLIEVLCGKVTEAVNEGEDYLYELAKSHIENGRLYDMVNPGLWKQMDPQSFKIFSEIAYCCLKAEPSERPSIDQILIQLEKALESQQEQENHQEKIEGATSKSLKLAHLQIPLEEIVLATNNFSDANLIGRDGFGKEYKGQLLRSHQSVNIVARRFLSDNSRRESKEFFMEVSMLSKLKHKNLVSIIGFCDENGEKIVINKHEIHGSLYHYLQDPTVTWMRRLQICVGAAHALSYIYYDKRRDFSIIHRNIKSSKILLDEQWEAKLSGFELSITQTASRRDRLCIPADVCGTIGYCDPTYIHSGIVTHKSDIYSFGIVLLEVMCGKPSYDDKLLAPSARSFYEEGRLEDMIDPVLWKQMDPKSFKIFSETAYYCVKEQRQQRPNIHQIVIQLEKALEFQWKHQNPDFISKAHSMVKGMTSKSLKGKNLKHLEIPLIDIQLATENFGKTYLIGSGGYGEVYKAELDHFDSKNLLPLEEKKTREHTKKRATVAIKRILNREDKQGIQGFLAEIEMLTNCKHPNIISLLGFCKEGDHMILVYEHASKGSLSDYLGSIGGKLTNLTWVQRIKICIDIARGLNYLHTKIEDDRRIIHRDIKSDNILLTENWQAKIADFGLSRLRSVKKSQQANKTLNTETLAGTDFYWDPEYFKTGRLKNESDIYSFGVVLFEILSGKLANDEIYTKENINGIAPVARRRFNEGTIREMVDPKLMEETHDNIFTSSRGPDKDSLDTFTKIAYQCVAESQTNRPTAEVIIKELEEALSLQENIKDHLKFSLDDIKTATQDFSHNNLIGHGGFGNVYKGEVTHAKGRSTIAAKRWHGEYEQGEHEFLMELQILLEYKHENCIGLLGYCNEMGEKIIAYEYASRGSLDRYLKDKGLMWMKRLEICIDIASGLDFLHGGGVAQEPVMHRDIKSSNVLLTNDWKGKISDFGLSLISPLNKDIDFVIDNLCGTIGYIDPLYLKLGFLTKESDIYSLGVVLFEILSGRDAYVDYNTHGHLSVLVKNNFNEGKLDEMVFEGIKEQIAPKSLATFQIIAYQCLHDDNKKRPTSRDVLLQLKKSLEFQEDYEIWERKLPNDYEQIFKMSKFSEIYSTEKKKDLHNLLSKGIILQKGTEWFLVGANGKRNQMISAWKFLYKQRQSYKWGYVPYSRFQKVAEMLDFSNLKIKMKIKTQLLSSGVNYGIHLIFRICGPRKSLAKQMYVNLKYKKGRGSETLHAYFATWREDDWMMIELCRFSNDKENIDLEFLLESLSRYYCGSHGIYVEGIEFRAIDNVKHEETDEIQEVQQVLKSDSNMNLMQQLPTNLEEIFKRSENEDGDEKLFLLNEVNGKKYLMLSAKAAVYNYSDVKLFKTKPSIQSRFLEVIELLPQQVLRINCKIKSQMLSRDTDYTCYLVFKLSEKCRGLHCPVKVRNLLHRNKKEVEIIYFRTPNAWNLHDTNQAPQQRPDGWMEVKVWKFNLNHELIKNDSFRVNLKLISYEGTLSGLILCGLEFRPL

SEQ ID NO: 7 - DNA, 5136 bp, Lactuca saligna
  misc_feature 1..5136: note = cDNA
  source 1..5136: mol_type = other DNA
atgtctccccacaaacagtttgatcacttacgaatcccgcttaaagagatagtatctgccaccgacaacttttctgatgcaaatttcatcagatatgatggatttggaaaggagtacaaaggacaactcttgcagtccggtcgattggtcgatatcgttgcacggaggttagatgataagaatggaataggaatcaaagagttttggatggaggtctcgatgctttgtacacttgagcatcaaaatatcgtctctatcgttgggttttgtgacgatggtgatgatggtgagaagatcgtcataaacaagcttgagacccgtggaagtcttcgtaactatctaagagacccgagcctcacatggatgcgaaggttgcagatatgtgttggtgttgcgcgggcattaaattacattcattatgatgagggacgtgattttagcgtcattcatcgcgatatcaagagttcaaagattttactagatgacaactgggaagcaaaattatatggttttgaactttccatgaatcaggcagcagctgaaaggaactgtccttgccagctatctagattttatggcacagagggatacatggacccgacatattttaagactggaagtgtgtcccacaagtccgatatgttttcctttggcgtggtattaattgaagtactttgtggcaaggtaacagaagctgtaaacgagggtgaggactatctatatgaattggccaaatcacatattgaaaatggaagactatatgatatggtgaatccgggtctatggaaacaaatggacccacaatcattcaaaatcttttcagaaatagcgtattgttgcttgaaggcagaaccatcagaacgaccaagtatagatcagattctcatacaacttgagaaagcactggaatctcaacaggaacaagaaaatcatcaagaaaagattgaaggcgcaacatccaagagcttgaagttggctcacttacaaatcccacttgaagagatagtattagccaccaacaacttttctgatgcaaaccttatcggacgtgatgggtttggaaaggagtacaaaggacaactcttgcggtcacatcaatcagtcaatattgttgcacggaggtttttaagtgataactcaaggagagaaagcaaagagttttttatggaggtttcgatgctttctaaacttaagcataaaaatcttgtctctatcattgggttttgtgatgagaacggtgagaagatcgtcataaacaaacatgagatccatggaagtctgtaccactatctacaagacccgaccgtgacatggatgcgaagattgcagatatgtgttggtgccgcgcacgctttgagttacatttattatgataagcgacgtgatttcagcatcatacaccgtaatatcaagagctccaagattttactggatgagcagtgggaagcaaaattatctggttttgaactttccatcactcagacagcaagtcgaagggaccgtctttgcatccctgcggatgtttgtggtacgatagggtactgcgacccaacatatattcatagtggaatcgtgacccacaagtcagatatttattcatttggcattgtattacttgaagtcatgtgtggaaagccttcctatgatgataagttgttagctccatcggccagatccttctatgaagagggaagactagaagatatgatagatccggttctatggaaacaaatggacccaaaatcattcaaaatcttttcggaaacagcatactactgcgtgaaggaacaacgacaacaacgtccaaatatacatcagattgtcatacaacttgagaaagccttagaatttcaatggaaacatcaaaaccctgatttcatatcaaaggctcactctatggttaagggcatgacttccaagagcttgaaggggaaaaacttgaaacacttagagatcccactcattgatatacaattggccaccgagaatttcggtaaaacatacctcattgggtcaggcggatatggtgaggtgtacaaagcagaacttgaccattttgatagcaaaaacttgttgccattagaagagaagaagacaagggaacataccaagaagcgcgccactgtagctataaaacgcatattgaatagagaagacaaacaaggaatacaagggttcctagcagaaattgaaatgcttacaaattgtaagcatcccaacataatctctcttcttggattttgcaaggaaggtgatcatatgatacttgtctacgagcatgcttctaaaggaagccttagtgattatttgggaagcatcggtggtaagttgactaatcttacttgggtgcaacgtataaagatatgtattgatattgcacgcggattgaattaccttcataccaagatagaggacgatcgaaggataatacaccgtgatataaaaagcgataacattttgttaactgagaattggcaagccaagattgctgattttgggctctccagattacgcagtgtgaaaaaaagtcaacaagcgaacaagactctcaatacagaaactcttgcaggcacagatttttattgggacccagaatattttaaaacgggtaggttgaaaaatgaatcagacatttactcttttggggtcgttttatttgagatcctgtcagggaagcttgccaatgatgaaatttacaccaaagaaaatatcaacgggattgcacctgtcgcaagacgtcgcttcaatgagggaacaataagggaaatggtagatcctaagttgatggaagaaactcatgacaacattttcacttcaagtagaggacccgacaaagattcattggatacatttacaaaaattgcataccaatgtgttgctgaatctcaaactaaccgtccaacagcagaagtcataattaaggaacttgaggaagcgttatctttacaagaaaacatcaaggaccacctcaaattctcacttgatgacattaaaactgccacacaagacttcagccataacaacttgattggtcatggaggatttgggaatgtatataaaggagaagttacacatgctaagggacgttctactattgctgcaaagcggtggcatggggaatatgaacagggagaacacgaatttttgatggagcttcaaattctcttggaatataaacatgaaaattgtattggtcttttaggctattgcaacgaaatgggtgaaaaaatcattgcttatgagtatgcttctagaggaagtctggataggtacttgaaggacaaaggtcttatgtggatgaaacggcttgagatatgcatcgatattgcaagcgggttggatttccttcatggaggtggagtagcacaagagccggtgatgcatagagacattaaaagctcaaacgttctattaaccaatgattggaaaggaaaaatttctgattttgggctttcgttaataagtccattaaataaggatattgactttgtcattgataatttatgcggcacaataggctatattgacccactatacctgaaattgggtttcttaaccaaagaatctgatatatattcactcggcgttgttttatttgagatattgtctggcagagatgcatatgttgattacaatacgcatgggcatctatctgtattggttaaaaacaacttcaatgaaggaaaacttgatgagatggtgttcgaaggtataaaagaacaaattgccccaaaatcattggctacatttcaaataattgcctaccaatgcttacatgatgataataaaaaacgaccaacatcacgcgatgtgctactacaactcaaaaagtcgttggaattccaagaggactacgaaatatgggagcgcaaattgcccaatgattatgaacaaatatttaaaatgtcaaaattttcggagatctactctactgaaaagaaaaaagacctccacaacttgctctccaaaggaatcatccttcaaaaaggcacagagtggtttttagtaggtgctaatggaaaaagaaatcaaatgatatcagcatggaagtttttatacaaacaacgccagtcatataagtggggatatgttccatactcaaggtttcaaaaggtagcagagatgctagatttttcgaatctaaagatcaaaatgaagataaaaactcaattattatcttcgggcgtcaattatgggattcatctcatctttagaatttgtggtccaagaaaatctctagctaagcagatgtacgtaaaccttaagtacaaaaaggggagagggagcgaaaccttacatgcatatttcgcaacatggagagaagatgattggatgatgattgaactgtgtcgattctcaaatgacaaggaaaatattgacttggagtttctgctagaaagcctttcgcgatactattgtggaagccatggcatatatgttgaaggcattgagtttcgagccattgataatgtgaaacatgaagaaacggatgaaatacaagaagtgcaacaagtcctgaaatcagactcaaacatgaatctcatgcaacaattgccaactaaccttgaagaaatatttaagagatctgaaaatgaggatggagatgaaaagctgtttttgctcaacgaagtgaacggaaagaaatatcttatgctttctgcaaaggctgctgtttacaactattctgatgtgaagcttttcaaaacaaaaccatcaattcaatccagatttctagaggtgattgagcttctaccacaacaagtacttcgtatcaattgcaagattaaaagtcaaatgttatcacgagatacagactacacatgttatcttgtgttcaagctttcagaaaagtgtcgtgggctgcattgtcctgtgaaagtaagaaatctacttcatcggaacaagaaagaggttgaaattatttattttagaaccccaaatgcatggaatttacatgatactaatcaagctccacaacaaaggccagatggatggatggaagttaaggtctggaaattcaacctaaatcatgaactcataaaaaatgatagttttcgtgtgaatttgaagttaattagttatgaagggactctatccggccttattttatgtggccttgaatttcggccactgtaa

SEQ ID NO: 8 - protein, 1711 aa, Lactuca saligna; identical to SEQ ID NO: 6
  REGION 1..1711: note = protein

SEQ ID NO: 9 - DNA, 5133 bp, Lactuca saligna
  misc_feature 1..5133: note = cDNA
  source 1..5133: mol_type = other DNA
atgtctccccacaaacagtttgatcacttacgaatcccgcttgaagagatagtatctgccaccgacaacttttctgatgcaaatttcatcagatatgatggatttggaaaggagtacaaaggacaactcttgcagtccggtcgattggtcgatatcgttgcacggaggttagatgataagaatggaataggaatcaaagagttttggatggaggtctcgatgctttgtacacttgagcatcaaaatatcgtctctatcgttgggttttgtgacgatggtgatgatggtgagaagatcgtcataaacaagcttgagacccgtggaagtctttgtaactatttaagagacccgagcctcacatggatgcgaaggttgcagatatgtgtcggtgttgcgcgggcattaaattacattcattatgatgagggacgtgattttagcgtcattcatcgcgatatcaagagttcaaagattttactagatgacaactgggaagcaaaattatatggttttgaactttccatgaatcaggcagcagctgaaaggaactgtccttgccagctatctggattttgtggcacagagggatacatggacccgacatattttaagactggaagtgtgtcccacaagtccgatatgttttcctttggcgtggtattaattgaagtactttgtggcaaggtaacagaagctgtaaacgagggtgaggactatctatatgaattggccaaatcacatattgaaaatggaagactatatgatatggtgaatccgggtctatggaaacaaatggacccacaatcattcaaaatcttttcagaaatagcgtatcgttgcttgaaggcagaaccatcagaacgaccaagtatagatcagattctcatacaacttgagaaagcactggaatctcaacaggaacaagaaaatcatcaagaaaagattgaaggcgcaatatccaagagcttgaagttggctcacttacaaatcccacttgaagagatagtatccgcctccaacaacttttctgatgcaaaccttatcggacgtgatgggtttggaaaggagtacaaaggacaactcttgcggtcacatgaattagtcaatattgttgcgcggaggttttttgatgataactcaaggagagaaagcaacaggttttttatggaggtttcgatgctttcaaaacttaagcatagaaatcttgtctctatcattgggttttgtgatgagaacggtgagaagatcgtcataaacaaacatgagatccatggaagtctgtacgactatctacaagacccgaccgtgacatggatgcgaagattgcagatatgtattggtgccgcgcacgctttgagttacatttattatgataagcgacgtgatttcagcatcatacaccgtgatatcaagagctccaagattttactggatgagcagtgggaagcaaaattatctggttttgaactttccatcactcagacagcaagtcgaagggaccgtctttgcattcctgcggatgtttgtggtacgatagggtaccgtgacccaacatatgttcagagtggaatcgtgacccacaagtcagatatttattcatttggcgttgtattatttgaagtcctgtgcggaaagcattgtgatgatcataagttgttagctccatcggccagatcatactatgaagagggaagactagaagatatgatagatccggttctatggaaacaaatggacccaaaatcattcaaaatcttttcggaaacagcatactactgcgtgaaggaacaacgacaacaacgtccaaatatacatcagattgtcatacaacttgagaaagccttggaatttcaatggaaacatcaaaaccctgatttcatatcaaaggctcactctatggttaagggcatgacttccaagagcttgaaggggaaagacttgaaacacttagagatcccactcattgatatacaattggccaccgagaatttcggtaaaacatacctcattgggtcaggcggatatggtgaggtgtacaaagcagaacttgaccattttgatagcaaaaacttgttgccattagaagagaagacaagggaacataccaagaagcgcgccactgtagctataaaacgtatattgaatagagaagacaaacaaggaatacaagggttcctagcagaaattgaaatgcttacaaattgtaagcatccgaacataatctctcttcttggattttgcaaggaaggtgatcatatgattcttgtctacgagcatgcttctaaaggaagccttagtgattatttgggaagcatcggtggtaagttgactaatcttacttgggtgcaacgtataaagatatgtattgatattgcacgcggattgaattaccttcataccaagatagaggacgatcgaaggataatacaccgtgatataaaaagcgataacattttgttaactgagaattggcaagccaagattgctgattttgggctctccagattacgcagtgtgaaaaaaagtcaacaagtgaacaagactctcaatacagaaactcgtgcaggcacagatttttattgggacccagaatattttaaaacgggtaggttgaaaaatgaatcagacatttactcttttggagtcgttttatttgagatcctgtcagggaagcttgccaatgatgcaatttacaccaaagaaaatatcaacgggattgcacctgtcgcaagacgtcgcttcaatgagggaacaataagggaaatggtagatcctaagctgatggaagaaactcatgacaacattttcacttcaagtagaggacccgacaaagattcattggatacatttacgaaaattgcatatcaatgtgtggctgcatctcaaactaaccgtccaacagcagaagtcataattaaggaacttgaggaagcgttatctttacaagaaaacatcaaggaccacctcaaattctcacttgatgacattaaaactgccacacaagacttcagtcataacaacttgattggccatggaggatttggggatgtatataaaggagatgttacacatgctaagggacgttctactattgctgcaaagcggttgcatgccagatatgatcagggagaacaccaatttttgatggagcttaaaattctcttggagtataaacatgaaaattgtattggtcttttaggctattgcaacgaaatgggtgaaaaaatcattgtttatgagtatgcttctagaggaagtctggataggtacttgaaggacaaaggtcttatgtggatgaaacggcttgagatatgcatcgatattgcaagtgggttggatttccttcatggaggtggagcggcacaagagccggtgatacatagagacattaatagctcaaacgttctattaaccaatgattggaaaggaaaaatttctgattttgggctttcgttaataagtccattaaataaggatattgactttgtcattgatgatttttgcggcacaataggctatattgacccactatacctgaaattgggtttcttaaccaaagaatctgatatatattcactcggcgttgttttatttgagatattgtctggcagagatgcatatgttgattacaatacgcatgggcatctatctgtattggttaaaaacaacttcaatgaaggaaaacttgatgagatggtgttcgaaggtataaaagaacaaattgccccaaaatcattggctacatttcaaataattgcctaccaatgcttacatgatgataataaaaaacgaccaacatcacgggatgtgctactacaactcaaaaagtcgttggaattccaggaggactacgaaatatgggagcgcaaattgcccaatgattatgaacaaatatttaaaatgtcaaaattttcggagatctactctactgaaaagaaaaaagacctccacaacttgctctccaaaggaatcatccttcaaaaaggcacagagtggtttttagtaggtgctaatggaaaaagaaatcaaatgatatcagcatggaagtttttatacaaacaacgccagtcatataagtggggatatgttccatactcaaggtttcaaaaggtagcagagatgctagatttttcgaatctaaagatcaaaatgaagataaaaactcaattattatcttcgggcgtcaattatgggattcatctcatctttagaatttgtggtccaagaaaatctctagctaagcagatgtacgtaaaccttaagtacaaaaaggggagagggagcgaaaccttacatgcatatttcgcaacatggagagaagatgattggatgatgattgaactgtgtcgattctcaaatgacaaggaaaatattgacttggagtttctgctagaaagcctttcgcgatactattgtggaagccatggcatatatgttgaaggcattgagtttcgagccattgataatgtgaaacatgaagaaacggatgaaatacaagaagtgcaacaagtcctgaaatcagactcaaacatgaatctcatgcaacaattgccaactaaccttgaagaaatatttaagagatctgaaaatgaggatggagatgaaaagctgtttttgctcaacgaagtgaacggaaagaaatatcttatgctttctgcaaaggctgctgtttacaactattctgatgtgaagcttttcaaaacaaaaccatcaattcaatccagatttctagaggtgattgagcttctaccacaacaagtacttcgtatcaattgcaagattaaaagtcaaatgttatcacgagatacagactacacatgttatcttgtgttcaagctttcagaaaagtgtcgtgggctgcattgtcctgtgaaagtaagaaatctacttcatcggaacaagaaagaggttgaaattatttattttagaaccccaaatacatggaatttacatgatactaatcaagctccacaacaaaggccagatggatggatggaagttaaggtctggaaattcaacctaaatcatgaactcataaaaaatgatagttttcgtgtgaatttgaagttaattagttatgaagggactctatccggccttattttatgtggccttgaatttcggccactgtaa

SEQ ID NO: 10 - protein, 1710 aa, Lactuca saligna
  REGION 1..1710: note = protein
MSPHKQFDHLRIPLEEIVSATDNFSDANFIRYDGFGKEYKGQLLQSGRLVDIVARRLDDKNGIGIKEFWMEVSMLCTLEHQNIVSIVGFCDDGDDGEKIVINKLETRGSLCNYLRDPSLTWMRRLQICVGVARALNYIHYDEGRDFSVIHRDIKSSKILLDDNWEAKLYGFELSMNQAAAERNCPCQLSGFCGTEGYMDPTYFKTGSVSHKSDMFSFGVVLIEVLCGKVTEAVNEGEDYLYELAKSHIENGRLYDMVNPGLWKQMDPQSFKIFSEIAYRCLKAEPSERPSIDQILIQLEKALESQQEQENHQEKIEGAISKSLKLAHLQIPLEEIVSASNNFSDANLIGRDGFGKEYKGQLLRSHELVNIVARRFFDDNSRRESNRFFMEVSMLSKLKHRNLVSIIGFCDENGEKIVINKHEIHGSLYDYLQDPTVTWMRRLQICIGAAHALSYIYYDKRRDFSIIHRDIKSSKILLDEQWEAKLSGFELSITQTASRRDRLCIPADVCGTIGYRDPTYVQSGIVTHKSDIYSFGVVLFEVLCGKHCDDHKLLAPSARSYYEEGRLEDMIDPVLWKQMDPKSFKIFSETAYYCVKEQRQQRPNIHQIVIQLEKALEFQWKHQNPDFISKAHSMVKGMTSKSLKGKDLKHLEIPLIDIQLATENFGKTYLIGSGGYGEVYKAELDHFDSKNLLPLEEKTREHTKKRATVAIKRILNREDKQGIQGFLAEIEMLTNCKHPNIISLLGFCKEGDHMILVYEHASKGSLSDYLGSIGGKLTNLTWVQRIKICIDIARGLNYLHTKIEDDRRIIHRDIKSDNILLTENWQAKIADFGLSRLRSVKKSQQVNKTLNTETRAGTDFYWDPEYFKTGRLKNESDIYSFGVVLFEILSGKLANDAIYTKENINGIAPVARRRFNEGTIREMVDPKLMEETHDNIFTSSRGPDKDSLDTFTKIAYQCVAASQTNRPTAEVIIKELEEALSLQENIKDHLKFSLDDIKTATQDFSHNNLIGHGGFGDVYKGDVTHAKGRSTIAAKRLHARYDQGEHQFLMELKILLEYKHENCIGLLGYCNEMGEKIIVYEYASRGSLDRYLKDKGLMWMKRLEICIDIASGLDFLHGGGAAQEPVIHRDINSSNVLLTNDWKGKISDFGLSLISPLNKDIDFVIDDFCGTIGYIDPLYLKLGFLTKESDIYSLGVVLFEILSGRDAYVDYNTHGHLSVLVKNNFNEGKLDEMVFEGIKEQIAPKSLATFQIIAYQCLHDDNKKRPTSRDVLLQLKKSLEFQEDYEIWERKLPNDYEQIFKMSKFSEIYSTEKKKDLHNLLSKGIILQKGTEWFLVGANGKRNQMISAWKFLYKQRQSYKWGYVPYSRFQKVAEMLDFSNLKIKMKIKTQLLSSGVNYGIHLIFRICGPRKSLAKQMYVNLKYKKGRGSETLHAYFATWREDDWMMIELCRFSNDKENIDLEFLLESLSRYYCGSHGIYVEGIEFRAIDNVKHEETDEIQEVQQVLKSDSNMNLMQQLPTNLEEIFKRSENEDGDEKLFLLNEVNGKKYLMLSAKAAVYNYSDVKLFKTKPSIQSRFLEVIELLPQQVLRINCKIKSQMLSRDTDYTCYLVFKLSEKCRGLHCPVKVRNLLHRNKKEVEIIYFRTPNTWNLHDTNQAPQQRPDGWMEVKVWKFNLNHELIKNDSFRVNLKLISYEGTLSGLILCGLEFRPL

SEQ ID NO: 11 - DNA, 121 bp, synthetic construct
  misc_feature 1..121: note = Marker 1
  source 1..121: mol_type = other DNA
tggtcatcgtgtaagcagatgtgacatggtgatgatgatgaaggtggtggtgtggttggcggtgtaaaagacgatgagtgcagcggtgcagattcatcaaagtcagaaaaaaccataacgg

SEQ ID NO: 12 - DNA, 121 bp, synthetic construct
  misc_feature 1..121: note = Marker 2
  source 1..121: mol_type = other DNA
gattagcctccatcttcctattttgaaaccctagcctctagcatgattgagagcttgtttgcttgattgaagagcttattctcggaatttggtgaagcttgaggaaagaggaagttcatat

SEQ ID NO: 13 - DNA, 121 bp, synthetic construct
  misc_feature 1..121: note = Marker 3
  source 1..121: mol_type = other DNA
atgggagaggtatgggatgcatcgactctgataactgtggtggatttcatctccgactccggcgatacctccccagagagaagataggggctcaaatgttaagttgccaatagaagataag

SEQ ID NO: 14 - DNA, 121 bp, synthetic construct
  misc_feature 1..121: note = Marker 4
  source 1..121: mol_type = other DNA
aatccagatgggtggtcaaaatcaaaatcaaaatcaaaatcaaagtcaaagtcaaggtcaaggtcatggtcagtcgattccgaatgctattcctcatcatgggatgaggcaacagccggtt

SEQ ID NO: 15 - DNA, 251 bp, synthetic construct
  misc_feature 1..251: note = VIGS1
  source 1..251: mol_type = other DNA
tgaaaattgtattggtcttttaggctattgcaacgaaatgggtgaaaaaatcattgcttatgagtatgcttctagaggaagtctggataggtacttgaaggacaaaggtcttatgtggatgaaacggcttgagatatgcatcgatattgcaagcgggttggatttccttcatggaggtggagtagcacaagagccggtgatgcatagagacattaaaagctcaaacgttctattaaccaatgattggaaag

SEQ ID NO: 16 - DNA, 250 bp, synthetic construct
  misc_feature 1..250: note = VIGS2
  source 1..250: mol_type = other DNA
atgtcttcccacaaacagtttgatcacttacgaatcccgcttgaagagatagtatctgccaccgacaatttttctgatgcaaatttcatcagacatgatggatttggaaaggagtacaaaggacaactcttgcagtccggtcgattggtcgatatcgttgcacgaaggttagatgataagaatggaataggaatcaaagagttttggatggaggtctcgatgctttttacacttgagcatcaaaatatcg

SEQ ID NO: 17 - DNA, 250 bp, synthetic construct
  misc_feature 1..250: note = VIGS control
  source 1..250: mol_type = other DNA
atggcgatggagggaagaagacatgaaaaggagagagacatcgatgatttgccgaaaaacgaagcaaactacacagcactaactccactttggtttttgaaaagagctgctctggttcacccccaccgtaggtcggttgtgcacggctccgttcagtacacatggcttcaaacttacctgcgttgctgccgattatcctccgctctttccaaacactccgtcggattcggttcaacggtagcagtgattg

(19) United States
(12) Patent Application Publication
(10) Pub. No.: US 2026/0022394 A1
(43) Pub. Date: Jan. 22, 2026

(54) LETTUCE PLANT RESISTANT TO DOWNY MILDEW AND RESISTANCE GENE

(71) Applicant: ENZA ZADEN BEHEER B.V., Enkhuizen (NL)

(72) Inventors: Bas Ter Riet, Medemblik (NL); Leonardus Cornelis Pieter Pijfers, Enkhuizen (NL); Marieke Maria Cornelia Wilhelmina Bos, Nijmegen (NL); Kim Anna Maria Lakeman, Hoorn (NL)

(21) Appl. No.: 18/996,434

(22) PCT Filed: Jul. 18, 2022
(86) PCT No.: PCT/EP22/70088

§ 371 Date: Jan. 17, 2025

(51) Int. Cl. C12N 15/82; C07K 14/415

(52) U.S. Cl. CPC C12N 15/8282; C07K 14/415

(57) ABSTRACT

The present invention relates to a lettuce plant that is resistant to downy mildew, more specifically to a lettuce plant that comprises a resistance gene that confers broad spectrum resistance to oomycetes in lettuce, more specifically B. lactucae. Furthermore. the present invention relates to a resistance gene and a method for obtaining a lettuce plant that is resistant to downy mildew, wherein the method comprises the step of introducing said resistance gene into a lettuce plant.

The present invention relates to a lettuce plant that is resistant to downy mildew, more specifically to a lettuce plant that comprises a resistance gene that confers broad spectrum resistance to oomycetes in lettuce, more specifically Bremia lactucae. Furthermore, the present invention relates to a resistance gene and a method for obtaining a lettuce plant that is resistant to downy mildew, wherein the method comprises the step of introducing said resistance gene into a lettuce plant.

Downy mildew refers to several types of oomycete microbes that are pathogens of plants. Downy mildew can originate from various species, but mainly of Peronospora, Plasmopara and Bremia. Downy mildew is a problem in many food crops, for example in lettuce caused by B. lactucae, affecting the production of this crop worldwide. Plants that are being affected include food crops such as brassicas (e.g. cabbage), grape, spinach, lettuce, onion, and cucumber. Downy mildew infection shows symptoms of discoloured areas on upper leaf surfaces in combination with white, grey or purple mould located on the lower side of the leaf facing the floor. Disease is spread from plant to plant by airborne spores.

Lettuce, mostly known as Lactuca sativa, but also including Lactuca species such as L. serriola, L. saligna or L. virosa, is a very important crop worldwide. Some of the most popular varieties available are Iceberg, Romaine, Butterhead, Batavia and Oakleaf. There are many plant pathogens that affect L. sativa, and some of the diseases caused by these pathogens are downy mildew, sclerotinia rot, powdery mildew, fusarium wilt of which the most important disease is lettuce downy mildew, which is caused by the B. lactucae, an oomycete pathogen that belong to Peronosporaceae. For some vegetable crops, such as lettuce, cultivars with resistance to downy mildew are available. However, the pathogen under pressure will mutate to break down the disease resistance and new disease resistance in crops is needed to control infection. Especially in lettuce the occurrence of downy mildew resistance is particularly complex as there are many different races, and new downy mildew resistant species emerging all the time, as found in European and the USA markets.

B. lactucae is a biotrophic pathogen that drastically reduces the yield and quality of lettuce. In lettuce, infection of B. lactucae result in yellow to pale green lesions that eventually become necrotic due to secondary pathogens leading to major crop losses. Fungicides can be used to control B. lactucae, but eventually B. lactucae becomes immune to these chemicals, because over time the pathogen also acquires resistance to fungicides. Furthermore, there are multiple lettuce varieties available that are resistant to B. lactucae but resistance is quickly overcome because new Bremia races develop rapidly. Currently there are 37 different B. lactucae races. Therefore, it is of the utmost importance to find other methods to control B. lactucae infection. Most preferably is to identify a resistance gene that gives broad resistance against B. lactucae and to provide for lettuce plants that are resistant to downy mildew.

Identification of new resistance genes is a promising alternative in the battle for providing disease resistant plants. At present two types of resistance genes against B. lactucae are known in lettuce, namely R-genes (NBS-LRR genes) and the MACPF-gene. The majority of disease resistance genes in plants encode nucleotide-binding site leucine-rich repeat proteins, also known as NBS-LRR proteins (encoded by R genes). During the development of new disease or pathogen resistant plants often traits are being combined, for example by introgression of a genetic locus comprising one or more resistance gene, thereby combining multiple resistance genes to combat the pathogen being able to overcome the resistance.

Considering the above, there is a need in the art to provide plants that are resistant to downy mildew and wherein plants have a broad-spectrum resistance against this pathogen. Furthermore, it is an object of present invention to provide novel resistance genes suitable for providing plants having a broad-spectrum downy mildew resistance, and to provide a method to obtain such downy mildew resistant plants.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a downy mildew resistant lettuce plant, wherein said lettuce plant comprises a FER resistance gene comprising one or more mutations, wherein the FER resistance gene encodes for a protein having at least 94% sequence identity, preferably at least 95%, more preferably at least 98%, most preferably 100% sequence identity with amino acid sequence of SEQ ID No. 2 and/or SEQ ID No. 4 providing downy mildew resistance, wherein said FER resistance gene provides resistance to Bremia lactucae races Bl:22 and Bl:23 EU, and Bl:1-9US. The downy mildew resistance conferring gene FER is a dominant resistance trait, and may be homozygous or heterozygous present in a downy mildew resistant lettuce plant. The resistance gene against B. lactucae has been found on chromosome 1. This FER resistance gene of the present invention gives preferably also resistance to B. lactucae races Bl:22, 23, 29, 31, 33, 34, 36 and 37 EU wherein said Bremia races have been characterized and classified according to the SEXTET code by IBEB (International Bremia Evaluation Board).

The FER resistance gene is unique, since it is not present in the L. sativa reference genome and is present in the L. serriola, and L. saligna genome i.e. this means a new resistance conferring gene has been added to L. sativa genome. This gene is initially picked up by fine mapping and VIGS experiments based on the L. saligna genome. At present two types of resistance genes against B. lactucae are known in lettuce, namely R-genes (NBS-LRR genes) and the MACPF-gene. The majority of disease resistance genes in plants encode nucleotide-binding site leucine-rich repeat proteins, also known as NBS-LRR proteins (encoded by R genes). These proteins are characterized by nucleotide-binding site (NBS) and leucine-rich repeat (LRR) domains as well as variable amino-and carboxy-terminal domains and are involved in the detection of diverse pathogens, including bacteria, viruses, fungi, nematodes, insects and oomycetes. However, for the first time a new resistance gene was identified in L. serriola and L. saligna, which is homologous to Arabidopsis thaliana Feronia (FER). The FER gene as such has been described earlier almost 15 years ago in Arabidopsis. However in Arabidopsis the FER gene is known to regulate cell elongation and fertility. FER is a receptor like kinases (RLK) and member of the

Catharanthus roseus receptor-like kinase 1-like (CrRLKIL) protein kinase subfamily. The function of FER in promoting plant growth and regulating fructification in dicotyledon Arabidopsis has been investigated. It was also reported that the Arabidopsis mutant FER disrupts the female gametophytic control of pollen tube reception. FER in A. thaliana is characterized by two malectin-like domains, a transmembrane domain, and an intracellular Ser/Thr kinase domain. However, surprisingly, we have found for the first time that the FER gene, identified in L. serriola and L. saligna, is a resistance gene in lettuce and provides resistance against Bremia disease, B. lactucae. FER acts like a sensor that detect if the plant is under attack of a pathogen, FER will subsequently activate, by phosphorylation, the plants downstream immune response in the lettuce, presumably via one or more pathways including H+-exchange pathway, Immune receptor complex pathway, and/or JA-pathway of the plants immune system, or a combination of these.

Combining and stacking the known resistance genes with the FER resistance gene will provide broad spectrum Bremia resistance to lettuce plants. To decrease the chances of the pathogen overcoming the resistance, as often seen with R genes, multiple R genes can be combined to enhance the durability of disease resistance. In lettuce, bremia resistance genes on chromosome 1 are present in the Major Resistance Cluster 1 (MRC1), which is comprised of two regions A and B. The downy mildew resistant lettuce plant of the present invention may further comprise one or more resistance genes located on chromosome 1 at a significant distance from the FER resistance gene or with R genes located at different linkage groups. As such, stacking of multiple resistance genes will enable broad and durable Bremia resistance in lettuce.

To demonstrate that the FER resistance gene provides Bremia resistance, this FER resistance gene was silenced by tobacco rattle virus (TRV)-based virus-induced gene silencing (VIGS) to induce susceptibility to B. lactucae infection in resistant L. sativa lines containing the FER resistance gene. With VIGS it was demonstrated that the FER resistance gene was associated with downy mildew resistance, since VIGS induced gene silencing was used to create Bremia susceptibility in resistant Lactuca accessions containing FER resistance. Resistant lettuce plants were transiently transformed with a silencing construct specific against the FER resistance gene which will result in the silencing of the resistance gene and as a consequence made the plant or plant organs susceptible to B. lactucae infection, thus by “removing” or silencing the FER resistance gene via virus induced gene silencing.

According to a preferred embodiment, the present invention relates to the lettuce plant, wherein said one or more mutations is selected from the group consisting of D678E, A704T, S708A, L756I, and T774I, preferably said mutations are D678E, A704T, S708A, L756I, and T774I, i.e. the FER resistance gene preferably comprises said five mutations. Sequence alignments between the FER gene of plants that are susceptible (i.e. SEQ ID No. 2) to Bremia and lettuce plants that are resistant showed that on specific positions in the FER protein mutations were present that are likely to provide the resistant phenotype in lettuce to Bremia.

Protein homology modelling was done on the FER resistance protein. The FER resistance gene encodes for a protein that is similar to tandem protein kinases, and consists of three pseudo kinase, one kinase, and one Phloem protein 2 (PP2)-like domain. Protein modelling confirmed that the kinase domain has a different function to the pseudo kinase domains and that the kinase domain is likely involved in disease resistance. The difference between FER genes from resistant and susceptible plants is that the FER gene that confers resistance show several mutations that differ between the resistant and susceptible plants; comprising the mutations D678E, A704T, S708A, L756I, and T774I, and all are present in a single domain comprised of amino acid 663 to 977 of the FER resistance protein, which is the only real kinase domain and is proposed to bind and inhibit NLRs. Each of these mutations seem contribute to significantly change the function of the FER protein resulting in the FER resistance protein. These residues seem to function in NLR binding and inhibition. The kinase domain is proposed to get phosphorylated and ubiquitinated. Pathogens release effectors to silence this immune response, these are recognized by plant

Nucleotide binding (NB) Leucine rich repeat (LRR) protein receptors (NLRs) to reinstate the hypersensitive response (HR) to kill infected cells. NLRs can be inhibited by tandem protein kinases, which are degraded upon effector recognition to activate the NLR.

According to another preferred embodiment, the present invention relates to the lettuce plant, wherein the FER resistance gene comprises a coding sequence having at least 95% sequence identity, preferably at least 97%, more preferably at least 98%, even more preferably at least 99%, most preferably 100% sequence identity with SEQ ID No. 5, SEQ ID No. 7, or SEQ ID No.9. Said FER resistance gene is preferably located on chromosome 1 of the lettuce plant.

According to yet another preferred embodiment, the present invention relates to the lettuce plant, wherein the FER resistance gene encodes for a protein comprising at least 95% sequence identity, preferably at least 97%, more preferably at least 98%, even more preferably at least 99%, most preferably 100% sequence identity with amino acid sequence of SEQ ID No. 6, SEQ ID No. 8, or SEQ ID No. 10.

According to a preferred embodiment, the present invention relates to the lettuce plant, wherein the lettuce plant is further resistant to one or more of B. lactucae races selected from the group consisting of races B1: 22, 23, 29, 31, 33, 34, 36 and 37 EU.

According to yet another preferred embodiment, the present invention relates to the lettuce plant, wherein the plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

According to a preferred embodiment, the present invention relates to the lettuce plant, wherein the FER resistance gene is at least heterozygous present in the lettuce plant, preferably homozygous present.

According to yet another preferred embodiment, the present invention relates to the lettuce plant, wherein the FER resistance gene is obtainable, derived, or originates from a lettuce plant of L. saligna or L. serriola. Most preferably the present invention relates to the lettuce plant, wherein the FER resistance gene is obtainable, derived, or originates from a lettuce plant deposited (NCIMB Ltd, Aberdeen, Scotland on 2 Mar. 2022) under number NCIMB 43942, NCIMB 43943 or NCIMB 43944, preferably NCIMB 43943. NCIMB 43942, NCIMB 43943, and NCIMB 43944 comprise the resistance gene comprises a coding sequence of SEQ ID No. 5, SEQ ID No.9 and SEQ ID No. 7, respectively. Alternatively, the FER resistance gene is obtainable, derived, or originates from a lettuce plant deposited (NCIMB Ltd, Aberdeen, Scotland on 7 Mar. 2022) under number NCIMB 43950.

The present invention, according to a second aspect, relates to seed of a lettuce plant of the present invention, comprising a FER resistance gene encoding a protein as described above. The seed comprises the FER resistance gene as described above.

According to a preferred embodiment, the present invention relates to a resistance gene that confers resistance to B. lactucae in lettuce plants, wherein the resistance gene encodes for a protein having at least 94% sequence identity, preferably at least 95%, more preferably at least 98%, most preferably 100% sequence identity with amino acid sequence of SEQ ID No. 2 or SEQ ID No. 4. The FER resistant gene is a dominant trait.

According to another preferred embodiment, the present invention relates to a resistance gene that confers resistance to B. lactucae in lettuce plants, wherein said resistance gene comprises a coding sequence of SEQ ID No. 5, SEQ ID No. 7, or SEQ ID No.9.

According to another preferred embodiment, the present invention relates to a resistance gene that confers resistance to B. lactucae in lettuce plants, wherein the FER resistance gene encodes for a protein comprising amino acid sequence of SEQ ID No. 6, SEQ ID No. 8, or SEQ ID No. 10.

According to yet another preferred embodiment, the present invention relates to a resistance gene that confers resistance to B. lactucae in lettuce plants, wherein the resistance gene provides resistance to at least Bremia lactucae races Bl:22 and Bl:23, and Bl:1-9US, in addition the resistance gene preferably further provides resistance to Bremia lactucae races Bl29, 31, 33, 34, 36, and Bl:37 EU.

According to yet another preferred embodiment, the present invention relates to the resistance gene that confers resistance to B. lactucae in lettuce plants, wherein the plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

The present invention, according to a further aspect, relates to a method for identifying (i) a downy mildew resistant lettuce plant of the present invention or (ii) a seed from said plant wherein the method comprises the step of establishing, in the genome of a plant or seed the presence of a FER resistance gene encoding a protein as defined above. The step of establishing, in the genome of the seed, the presence of any genetic information, including the presence of the FER resistance gene encoding the protein as defined above, may suitably involve allowing the seed to grow into a plant and establishing the presence of the genetic information in the genome of the plant grown from the seed.

According to yet another preferred embodiment, the present invention relates to the method for identifying a downy mildew resistant lettuce plant of present invention, wherein the step of establishing comprises establishing the presence of SEQ ID No. 5, SEQ ID No. 7, SEQ ID No.9, and/or, wherein the step of establishing, comprises establishing the presence of one or more sequences selected from the group consisting of SEQ ID No. 11 in combination with SEQ ID No.

12, or SEQ ID No.13 in combination with SEQ ID No. 14.

A downy mildew plant of present invention comprising the FER resistance gene can be identified by the presence in the genome of said plant of one or more sequences selected from the group consisting of SEQ ID No. 5, SEQ ID No. 7, or SEQ ID No.9. Most preferably the plant can be identified by the presence of marker sequences SEQ ID No.11 in combination with SEQ IDs No. 12 or SEQ ID No.13 in combination with SEQ IDs No. 14.

The present invention, according to a further aspect, relates to a method for obtaining a lettuce plant that is resistant to downy mildew, wherein the method comprises the steps of,
a) crossing a lettuce plant comprised of the resistance gene of the present invention with a lettuce plant susceptible to downy mildew and which does not comprise said resistance gene,
b) optionally, selfing the plant obtained in step a) for at least one time,
c) selecting the plants that are resistant to downy mildew.

A plant having this resistant phenotype can be obtained via use of gene editing and/or mutation techniques, such as EMS mutagenesis or CRISPR/Cas in concert with cloning techniques on the FER resistance gene to generate disease resistant crops. A resistance gene can be brought into the plant by known means including e.g. transgenic techniques or by introgression, wherein the resistance providing sequence(s) are introduced into the plant.

The present invention, according to a further aspect, relates to the use of a gene construct or plasmid for introducing a resistance gene into the genome of a plant or plant cell and providing resistance to downy mildew caused by one or more of B. lactucae selected from the group of race Bl:22, and Bl:23EU, and Bl:1-9US, wherein the gene construct is comprised of the FER resistance gene as described above operably linked to expression providing sequences in said plant. The resistance gene of present invention may be transferred (e.g. by transformation or transfection) into plants, such as lettuce plants, using a plasmid or vector or linear gene construct that comprises the resistance gene of present invention. The FER resistance gene, after being transferred into the lettuce plant would provide resistance to B. lactucae, i.e. resistance to at least B. lactucae of race Bl:22, and Bl:23 EU, and preferably also Bremia lactucae races Bl:29, 31, 33, 34, 36 and 37EU.

The present invention will be further detailed in the following examples and figures wherein:

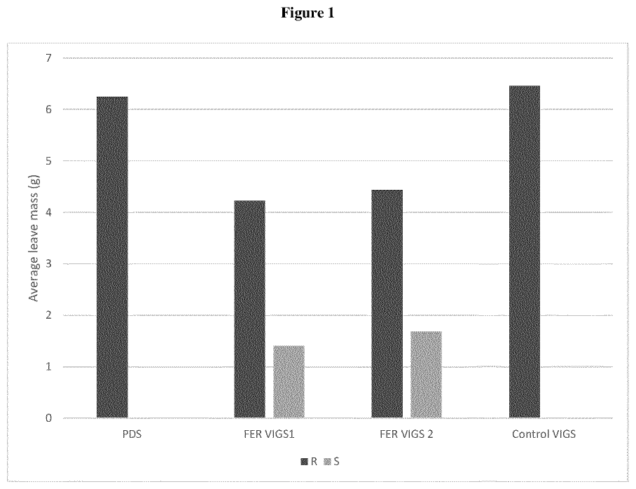

FIG. 1: shows the average weight of the susceptible and resistant leaves of lettuce that have been infected with Bremia lactucae (Bl:22), after VIGS silencing of the FER resistance gene of present invention of a lettuce plant of present invention comprising the FER resistance gene using VIGS gene silencing constructs of Table 2 and subsequently infected with B. lactucae. The leaves from plants wherein the FER resistance gene has been silenced by VIGS silencing (VIGS1 or VIGS2 silencing construct), showed the presence of susceptible leaves when infected with Bremia as compared to plants where the FER gene was not silenced (i.e. by VIGS control or PDS silencing construct on the plant comprising the FER gene). No susceptible leaves were detected in the control groups.

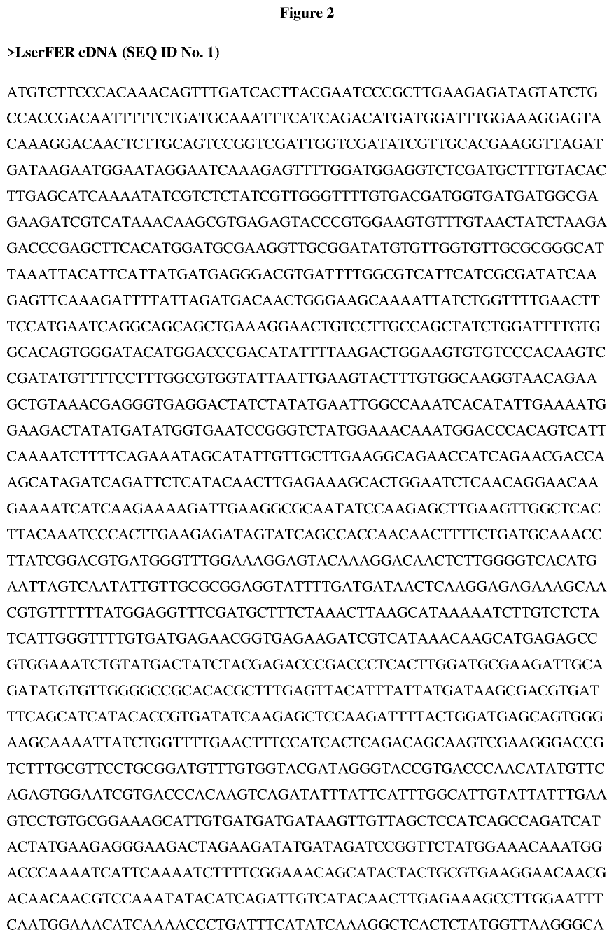

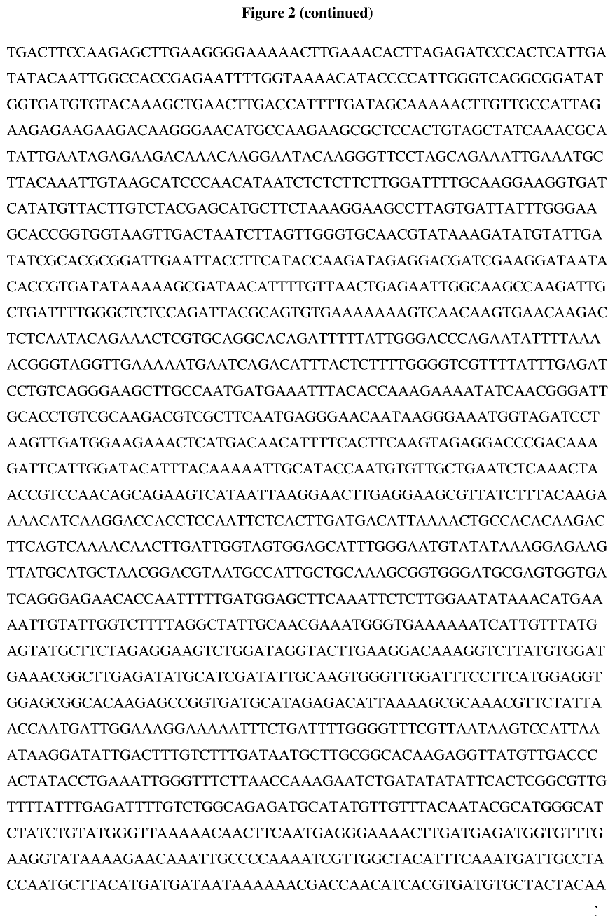

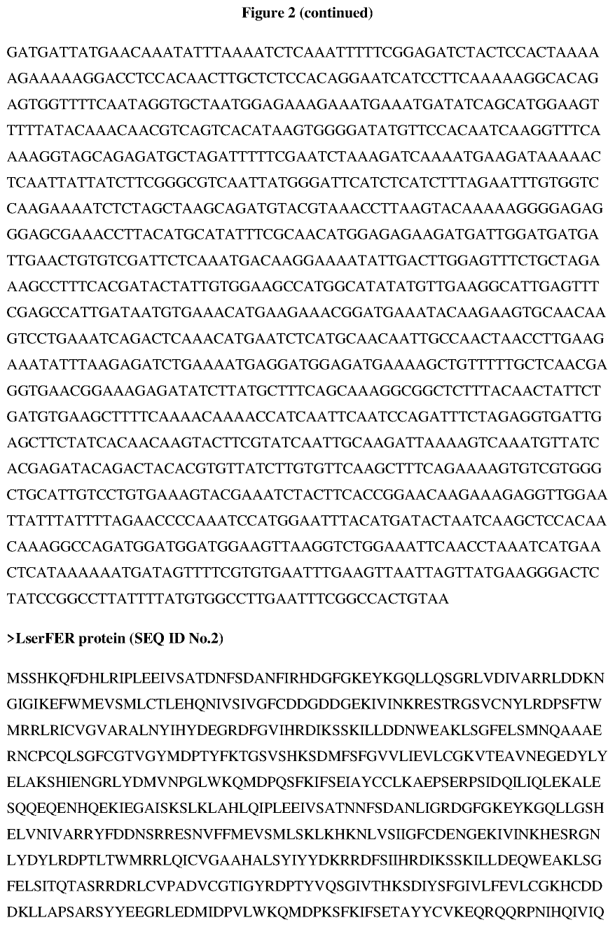

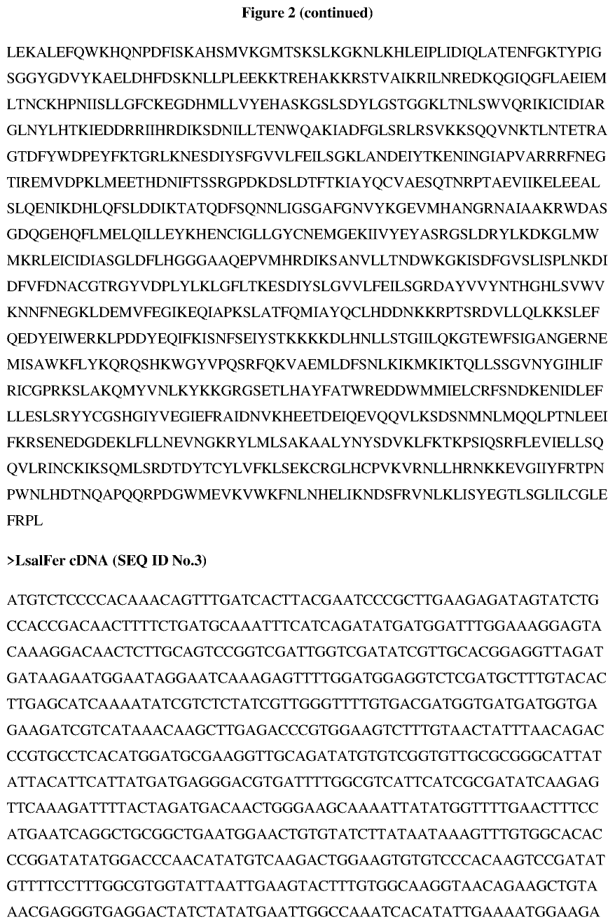

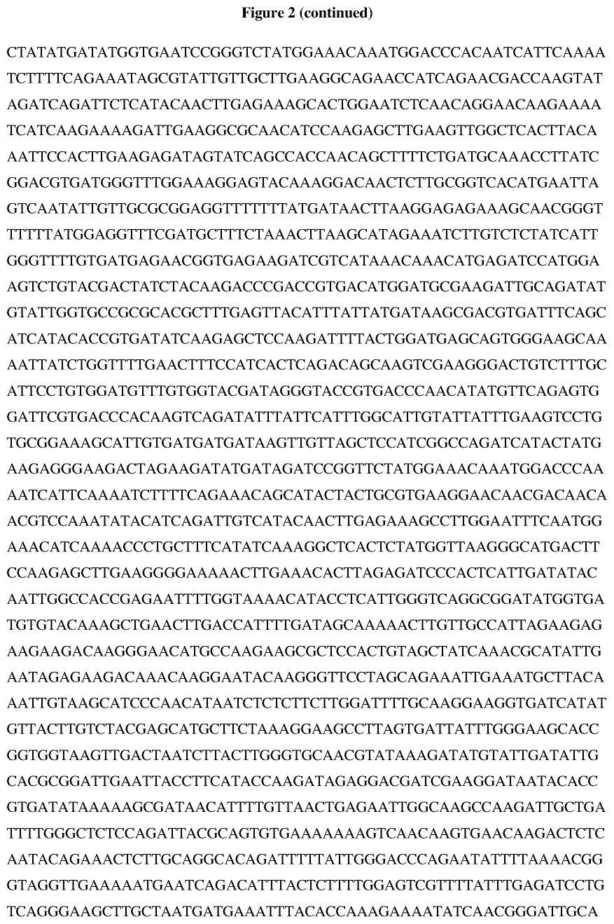

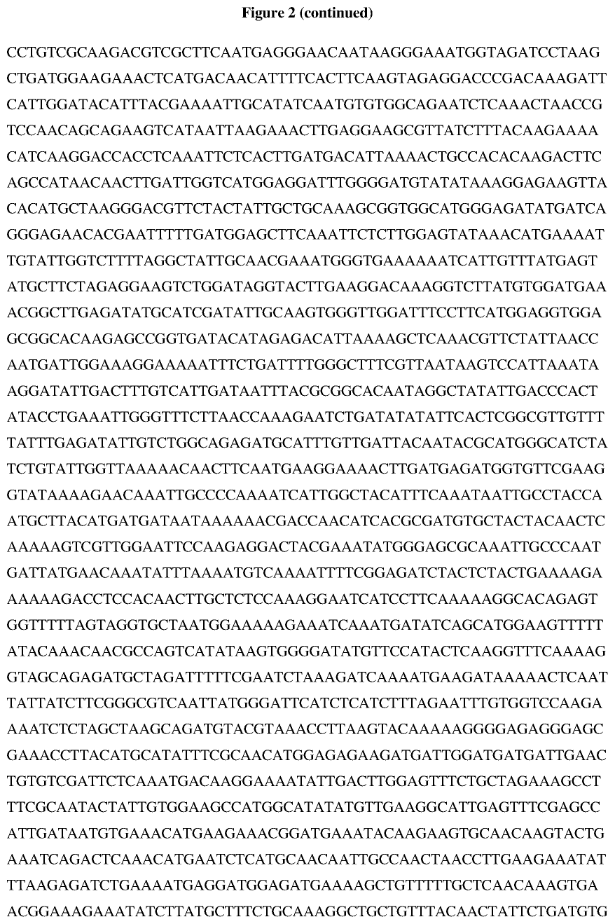

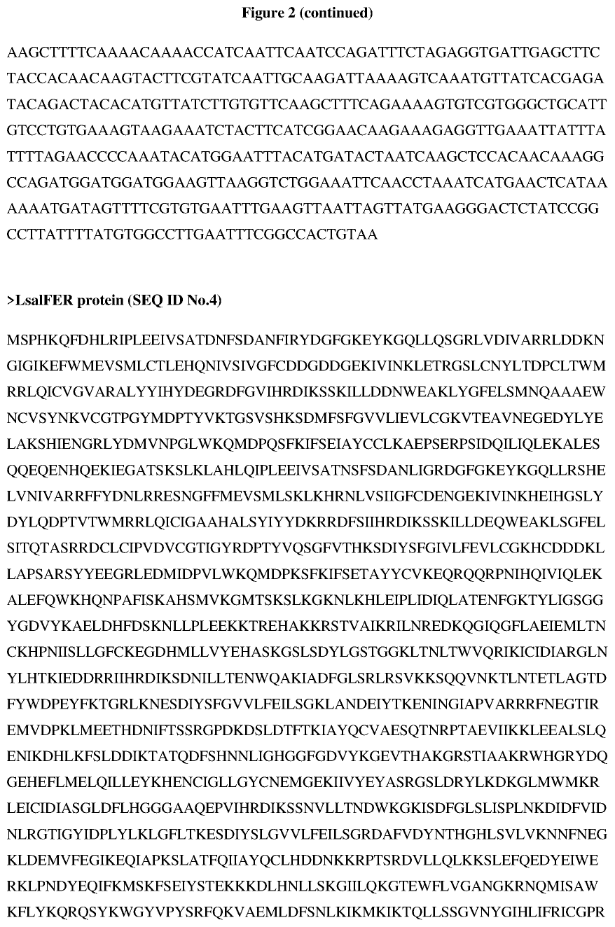

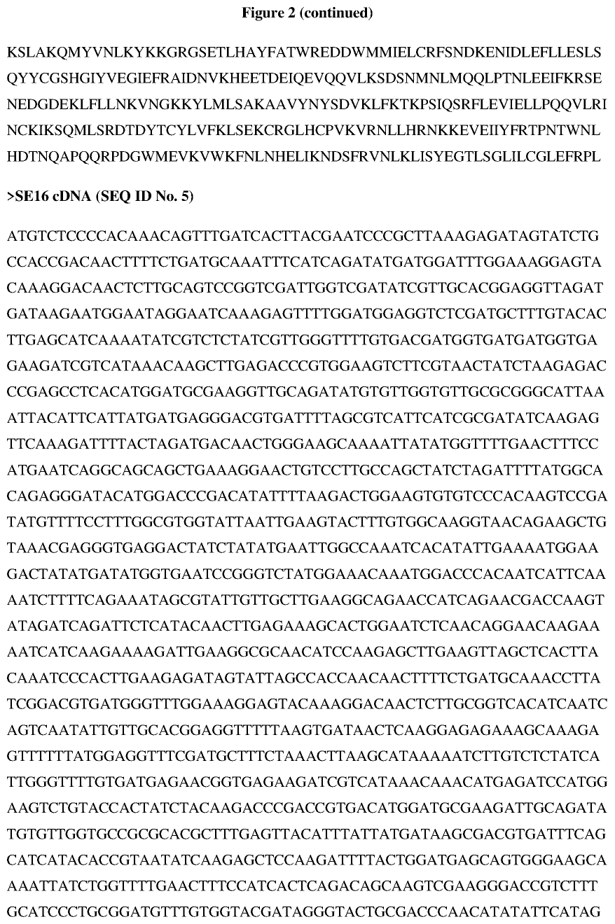

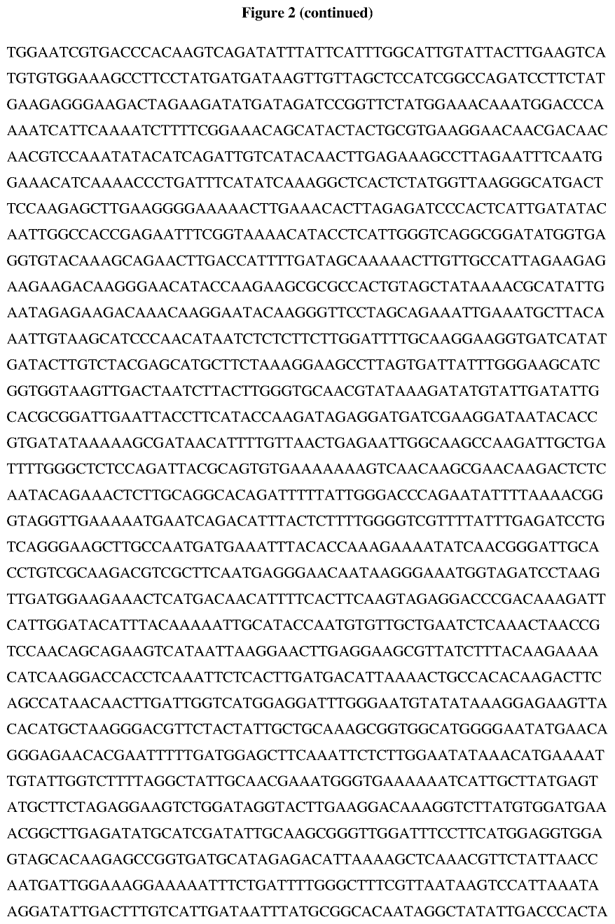

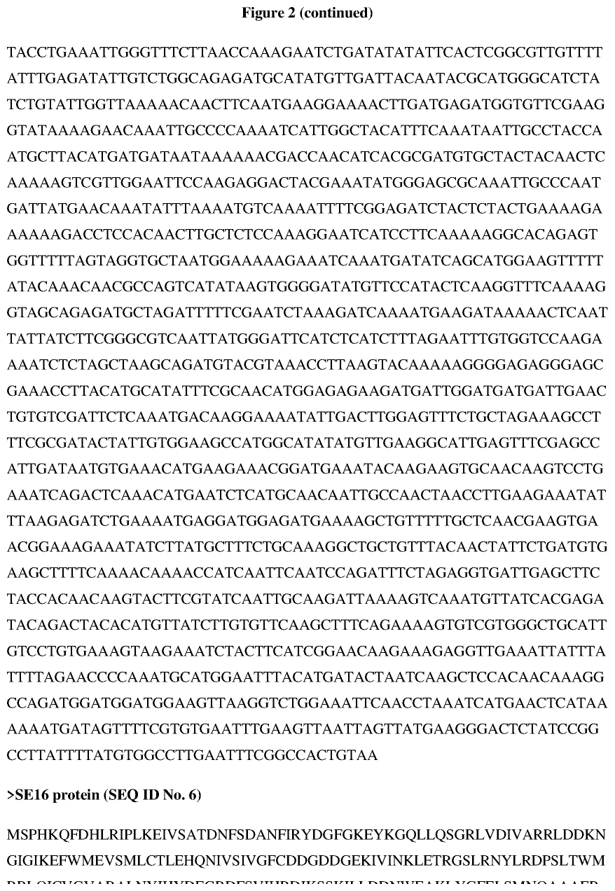

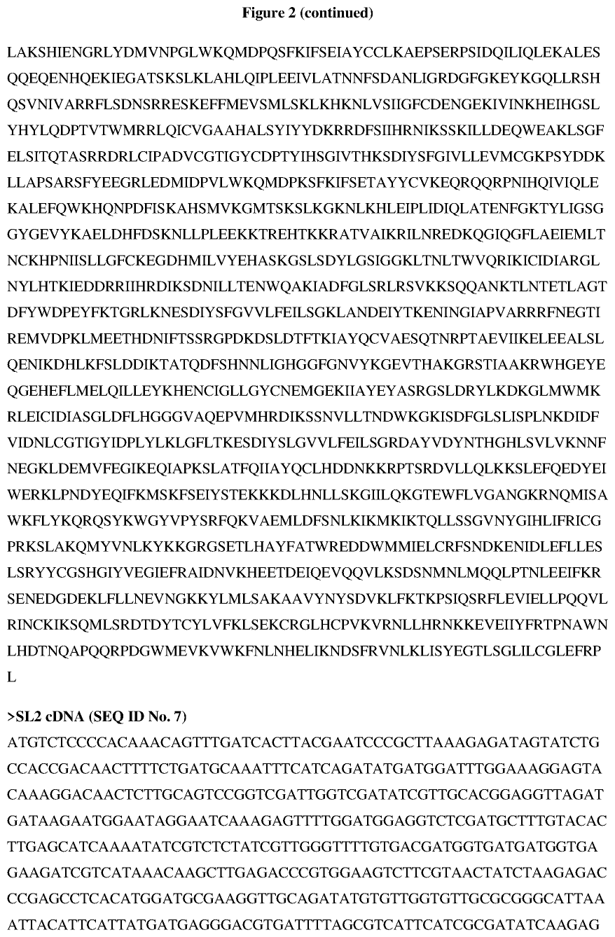

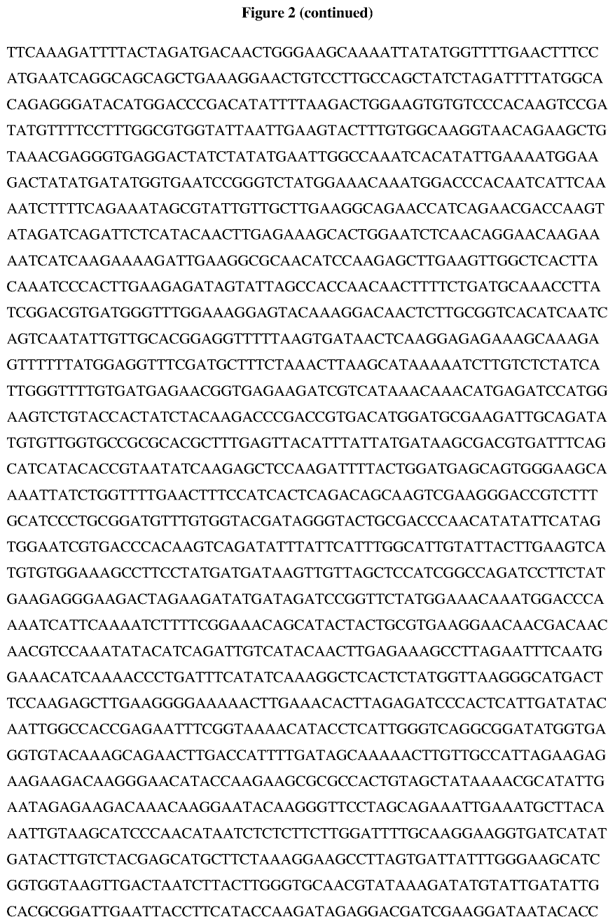

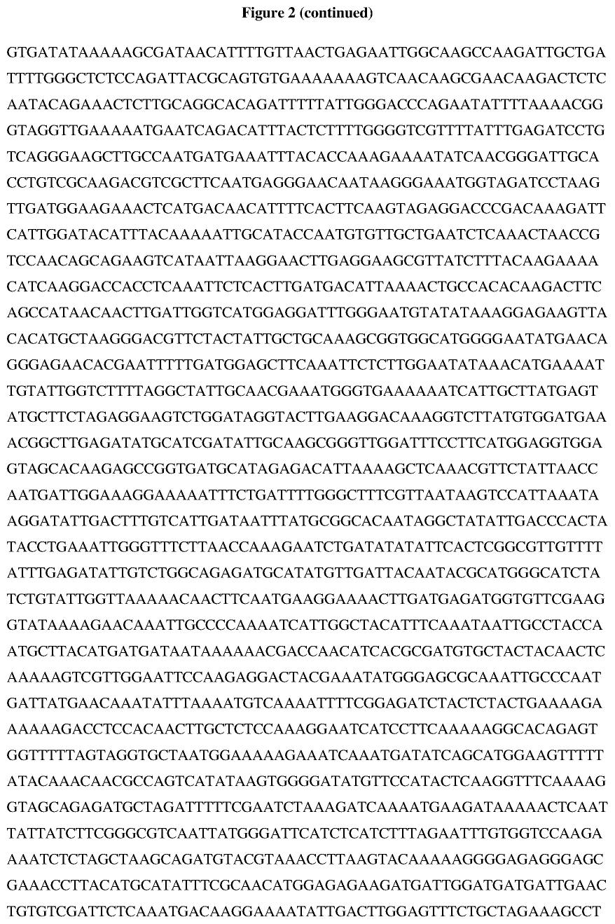

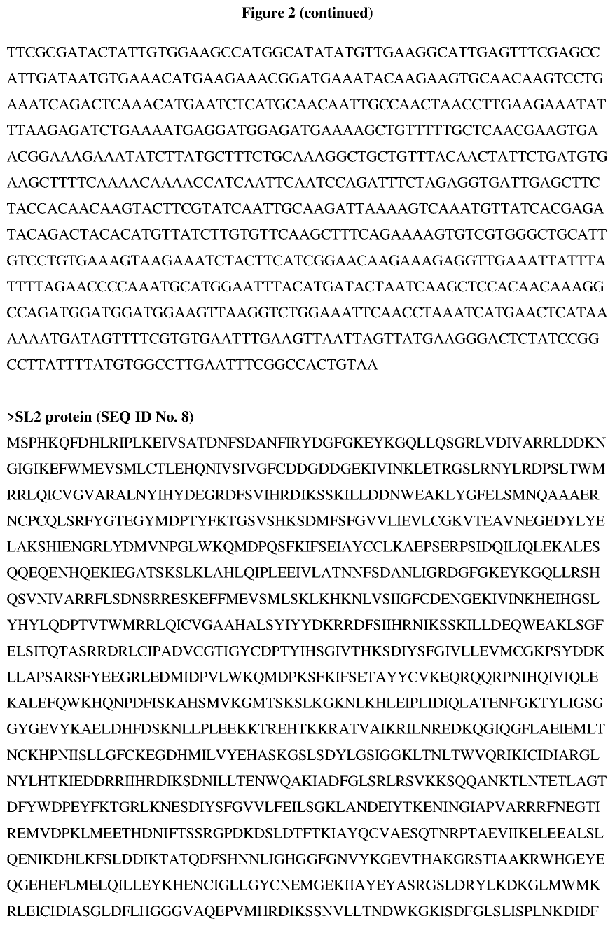

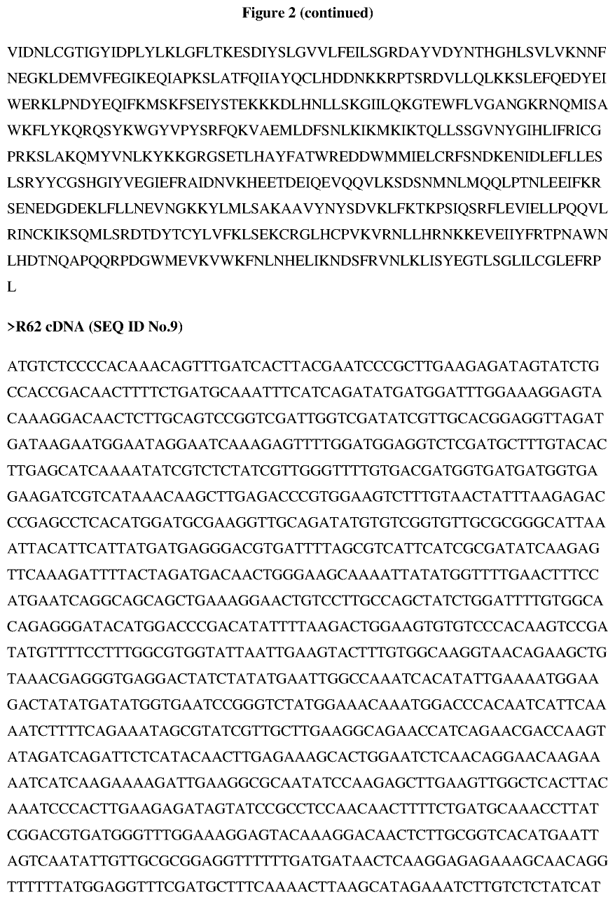

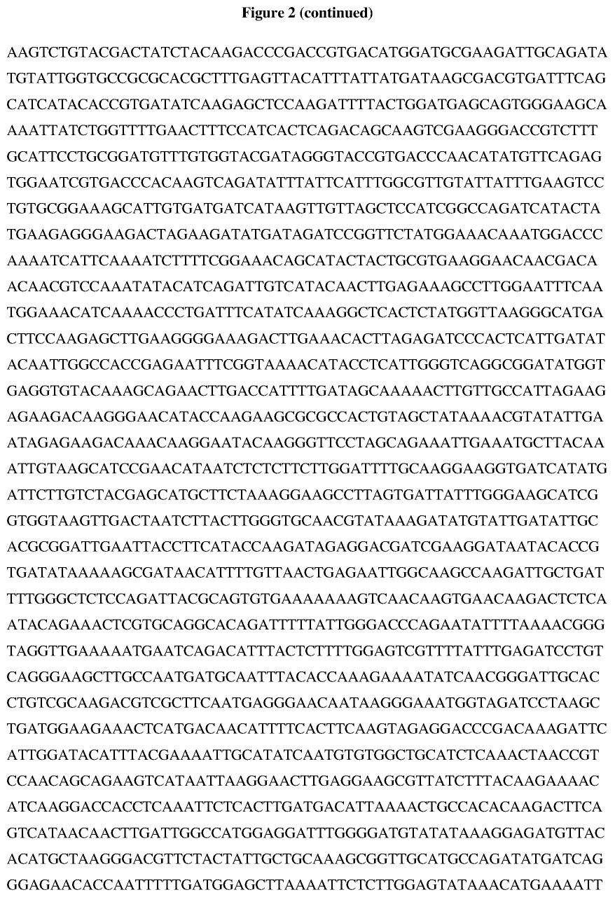

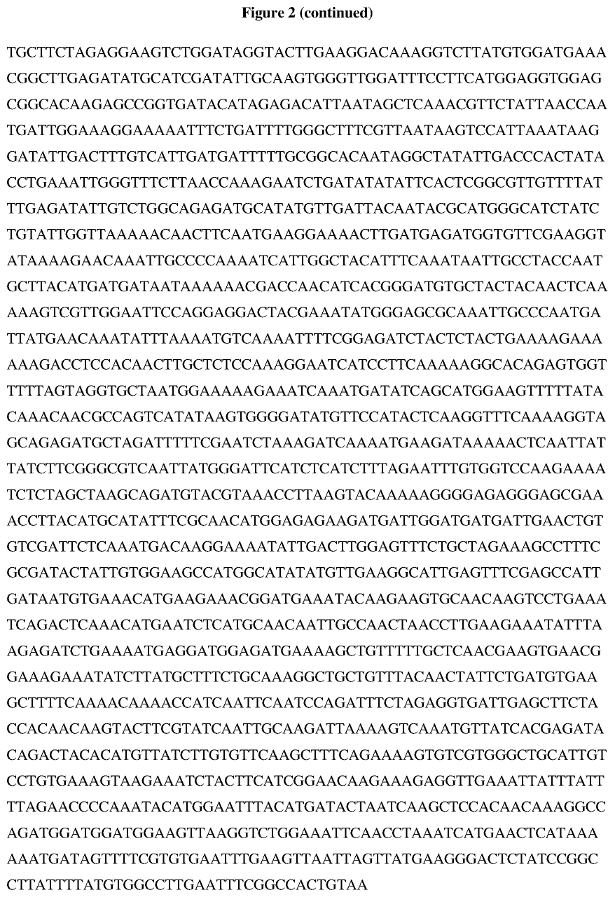

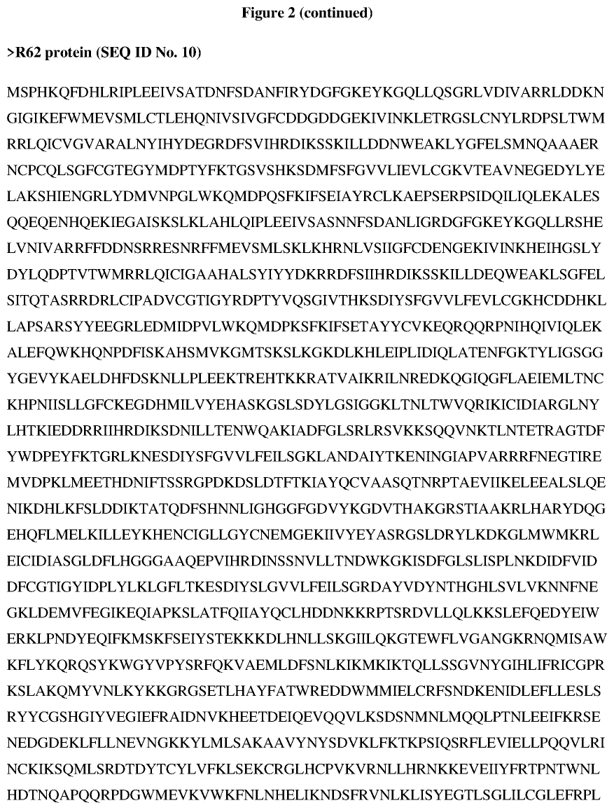

FIG. 2: shows the coding sequences (SEQ ID No. 1 and 3) and protein sequence (SEQ ID No. 2 and 4) of the FER gene from lettuce (L. serriola and L. saligna, respectively) susceptible to Bremia (B. lactucae). Furthermore, the coding sequences (SEQ ID No. 5, 7 and 9) and protein sequences (SEQ ID No. 6, 8 and 10) of the FER gene are shown that provide resistance to Bremia in lettuce.

### EXAMPLES

### Gene Mapping of FER Resistance Gene in L. saligna and L. serriola

Gene mapping experiments were done to identify a resistance gene that is involved in Bremia (B. lactucae) resistance in lettuce (L. sativa). The resistance gene was originally isolated from L. saligna and was mapped on chromosome 1. Furthermore, a similar resistance gene was isolated from L. serriola and was also mapped in the same region on chromosome 1. Interestingly in both cases there was no R gene (NBS LRR gene) identified in this region, however the new gene was identified as being a protein receptor kinase.

After fine mapping in a population of about 12,000 plants there was one putative resistance gene present in the identified resistance locus, identified as the FER gene. The identified resistance region that originated from L. serriola comprised marker 1 (SEQ ID No.11) and marker 2 (SEQ ID No.12). The identified resistance region that originated from L.saligna comprised marker 3 (SEQ ID No.13) and marker 4 (SEQ ID No.14). The SNPs are indicated in bold and underlined. In susceptible plants the indicated SNP nucleotide was an “A” in respect to SEQ ID No 11, 12 and 13, and a “T” for SEQ ID No. 14.

TABLE 1
Marker sequences

| Marker | Sequence |
|---|---|
| Marker 1 (SEQ ID | TGGTCATCGTGTAAGCAGATGTGACATGGTGATGATGATGAAGG |
| No. 11) | TGGTGGTGTGGTTGGCGGTGTAAAAGACGATGAGTGCAGCGGT |
| | GCAGATTCATCAAAGTCAGAAAAAACCATAACGG |
| Marker 2 (SEQ ID | GATTAGCCTCCATCTTCCTATTTTGAAACCCTAGCCTCTAGCATG |
| No. 12) | ATTGAGAGCTTGTTTGCTTGATTGAAGAGCTTATTCTCGGAATTT |
| | GGTGAAGCTTGAGGAAAGAGGAAGTTCATAT |
| Marker 3 (SEQ ID | ATGGGAGAGGTATGGGATGCATCGACTCTGATAACTGTGGTGGA |
| No. 13) | TTTCATCTCCGACTCCGGCGATACCTCCCCAGAGAGAAGATAGG |
| | GGCTCAAATGTTAAGTTGCCAATAGAAGATAAG |
| Marker 4 (SEQ ID | AATCCAGATGGGTGGTCAAAATCAAAATCAAAATCAAAATCAA |
| No. 14) | AGTCAAAGTCAAGGTCAAGGTCATGGTCAGTCGATTCCGAATGC |
| | TATTCCTCATCATGGGATGAGGCAACAGCCGGTT |

### FER Resistance Gene Silencing Experiment using Virus Induced Gene Silencing (VIGS)

To demonstrate that the FER resistance gene of L. serriola provides Bremia resistance, the FER resistance gene was silenced by tobacco rattle virus (TRV)-based virus-induced gene silencing (VIGS) to induce susceptibility to B. lactucae infection in L. sativa lines containing the FER resistance gene of L. serriola. Tobacco rattle virus (TRV)-derived VIGS vectors have been abundantly described to study gene function in Arabidopsis thaliana, Nicotiana benthamiana, Solanum esculentum and other plants (see for example Huang C, Qian Y, Li Z, Zhou X.: Virus-induced gene silencing and its application in plant functional genomics. Sci China Life Sci. 2012;55(2):99-108). With VIGS it was demonstrated that the FER resistance gene was associated with downy mildew resistance, since VIGS induced gene silencing was used to create Bremia susceptibility in resistant Lactuca accessions comprising the FER gene. Resistant lettuce plants were transiently transformed with a silencing construct specific against the resistance FER gene which will result in the silencing of the resistance gene.

Briefly, lettuce plants containing the FER resistance gene were silenced for FER resistance gene by VIGS using different silencing construct to identify if this FER resistance gene was indeed responsible for the observed resistance. Three VIGS-constructs were used, two (VIGS1 and VIGS2) that were designed for specific silencing of the FER resistance gene and VIGS control that silenced a region that is located next to the FER gene on the same chromosome, that served as a control. Furthermore, independent of resistance gene silencing the PDS gene is silenced as well that serves as positive control to indicate if VIGS is working and to determine the efficiency. The PDS gene is involved in carotenoid biosynthesis and is the first step in lycopene biosynthesis. This step is catalyzed by the enzyme phytoene desaturase (PDS). When silencing of the PDS gene is achieved, this results in bleached leaves. Experiments showed bleached leaves indicating that the VIGS silencing was achieved and performed correctly (data not shown). All plants that were VIGS inoculated were harvested and put in a tray and sprayed with Bremia to test the effect of the gene silencing on disease resistance.

The VIGS constructs as described above were cloned in the K20 vector (See Table 2 for sequences, SEQ ID No. 15, SEQ ID No. 16, SEQ ID No. 17). The constructs were transformed and transiently expressed into a lettuce plant of present invention that is resistant to Bremia, using co-cultivation with agrobacterium (GV3101) to study the resistance gene function in relation to Bremia resistance. The % of susceptible Bremia leaves was observed in both groups and both silencing constructs. With the leaves of VIGS-experiments independent disease tests (see below) were performed to observe that when FER resistance gene was silenced, plants became susceptible to Bremia.

Results (FIG. 1) indicate that when FER gene was silenced by VIGS with the VIGS1 or VIGS2 construct the plants became susceptible after Bremia infection (Bl22) confirming that the resistance gene is linked to a resistance gene that provides the plant resistance against Bremia.

TABLE 2
VIGS constructs

| VIGS-constructs | Sequence |
|---|---|
| VIGS1 | TGAAAATTGTATTGGTCTTTTAGGCTATTGCAACGAAATGGGTG |
| (SEQ ID No. 15) | AAAAAATCATTGCTTATGAGTATGCTTCTAGAGGAAGTCTGGAT |
| | AGGTACTTGAAGGACAAAGGTCTTATGTGGATGAAACGGCTTGA |
| | GATATGCATCGATATTGCAAGCGGGTTGGATTTCCTTCATGGAG |
| | GTGGAGTAGCACAAGAGCCGGTGATGCATAGAGACATTAAAAG |
| | CTCAAACGTTCTATTAACCAATGATTGGAAAG |
| VIGS2 | ATGTCTTCCCACAAACAGTTTGATCACTTACGAATCCCGCTTGA |
| (SEQ ID No. 16) | AGAGATAGTATCTGCCACCGACAATTTTTCTGATGCAAATTTCA |
| | TCAGACATGATGGATTTGGAAAGGAGTACAAAGGACAACTCTT |
| | GCAGTCCGGTCGATTGGTCGATATCGTTGCACGAAGGTTAGATG |
| | ATAAGAATGGAATAGGAATCAAAGAGTTTTGGATGGAGGTCTC |
| | GATGCTTTTTACACTTGAGCATCAAAATATCG |
| VIGS control | ATGGCGATGGAGGGAAGAAGACATGAAAAGGAGAGAGACATC |
| (SEQ ID No. 17) | GATGATTTGCCGAAAAACGAAGCAAACTACACAGCACTAACTC |
| | CACTTTGGTTTTTGAAAAGAGCTGCTCTGGTTCACCCCCACCGTA |
| | GGTCGGTTGTGCACGGCTCCGTTCAGTACACATGGCTTCAAACT |
| | TACCTGCGTTGCTGCCGATTATCCTCCGCTCTTTCCAAACACTCC |
| | GTCGGATTCGGTTCAACGGTAGCAGTGATTG |

### Disease Test and Biotest for Downy Mildew in LETTUCE

Leaves of resistant plants transiently transformed with the above described VIGS constructs, were put in trays with moistened paperboard and infected with Bremia. Infected seedlings are suspended in 20 mL water, filtered by cheesecloth and the flow-through is collected in a spray flask. The trays are spray-inoculated with the B. lactucae suspension. The trays are covered with a glass plate and stored in a climate chamber at 15°° C. (12 hours of light). A black, opaque foil is placed over the trays for one day to improve growth of B. lactucae. After one day, the foil is removed. Experiments were performed in triple, and eight to ten days after infection leaves are phenotypically scored by eye on the presence of Bremia, i.e. being susceptible or resistant (FIG. 1).

Disease resistance tests show that the FER resistance gene (SEQ ID NO: 9) from L. saligna provides resistance to Bremia races from Bl:16 to 23, Bl:29, 31, 33, 34, 36 and Bl:37EU (see Table 3). Furthermore, disease resistance test show that the FER resistance gene further provides resistance to US Bremia races Bl:1 to Bl:9 (results not shown). Table 3 shows an overview of the disease test performed with the most recent isolates of B. lactucae on L. sativa lines Cobham Green R273, DM3 line (both control lines), and the plant of present invention comprising the FER resistance gene. For the resistance gene of L. serriola a similar spectrum was observed.

TABLE 3
Disease resistance test Bremia races, (−) is resistant, (+) is susceptible.

| | Bl:16 | Bl:17 | Bl:18 | Bl:19 | Bl:20 | Bl:21 | Bl:22 | Bl:23 | Bl:24 | Bl:25 | Bl:26 |
|---|---|---|---|---|---|---|---|---|---|---|---|
| Cobham | + | + | + | + | + | + | + | + | + | + | + |
| Green | | | | | | | | | | | |
| DM3 | + | + | − | + | + | + | − | + | − | − | + |
| FER | − | − | − | − | − | − | − | − | nd | nd | nd |
| | Bl:27 | Bl:28 | Bl:29 | Bl:30 | Bl:31 | Bl:32 | Bl:33 | Bl:34 | Bl:35 | Bl:36 | Bl:37 |
| Cobham | + | + | + | + | + | + | + | + | + | + | + |
| Green | | | | | | | | | | | |
| DM3 | + | − | − | − | + | + | − | − | − | + | + |
| FER | nd | nd | − | nd | − | nd | − | − | nd | − | − |

A single gene line comprising the FER resistance gene was used internally to test Bremia diagnostic. Seeds of this line are deposited at NCIMB Ltd, Aberdeen, Scotland on 2 Mar. 2022, reference NCIMB 43943.

### Alignment of FER Resistance Gene Coding Sequences and Protein Sequences

The similarity of the FER resistance gene coding sequences (Table 4) and novel resistance proteins (Table 5) was determined using multiple alignment software. The FER coding sequence and protein sequence of a susceptible L. serriola (SEQ ID NO: 1 and 2, respectively), a susceptible L. saligna (SEQ ID NO: 3 and 4, respectively), a resistant L. serriola (SEQ ID NO: 5 and 6, respectively) and a resistant L. saligna (SEQ ID NO: 7 and 8, respectively) and another resistant L. saligna (SEQ ID NO: 9 and 10, respectively) were used to generate the results shown in Table 4 (cDNA sequence) and Table 5 (protein sequence). All FER resistance genes were highly similar at both the nucleotide and amino acid level; at least 96% identity on nucleotide level and at least 94% identity on amino acid level.

TABLE 4
Percent Identity Matrix of coding sequences of the FER genes

| SEQ ID No. | 1 | 3 | 5 | 7 | 9 |
|---|---|---|---|---|---|
| 1 | 100 | 96.8 | 97.0 | 97.1 | 97.2 |
| 3 | 96.8 | 100 | 98.0 | 98.0 | 98.3 |
| 5 | 97.0 | 98.0 | 100 | 99.96 | 98.4 |
| 7 | 97.1 | 98.0 | 99.96 | 100 | 98.4 |
| 9 | 97.2 | 98.3 | 98.4 | 98.4 | 100 |

TABLE 5
Percent Identity Matrix of amino acid
sequences of the FER proteins

| SEQ ID No. | 2 | 4 | 6 | 8 | 10 |
|---|---|---|---|---|---|
| 2 | 100 | 94.9 | 94.7 | 94.7 | 95.4 |
| 4 | 94.9 | 100 | 96.4 | 96.4 | 97.1 |
| 6 | 94.7 | 96.4 | 100 | 100 | 97.1 |
| 8 | 94.7 | 96.4 | 100 | 100 | 97.1 |
| 10 | 95.4 | 97.1 | 97.1 | 97.1 | 100 |

The multiple sequence alignment of the amino acid sequence of the FER proteins of susceptible and resistant lettuce as indicated above, showed that the FER resistant protein comprised specific mutations at position 678 (D→E), 704 (A→T), 708 (S→A), 756 (L→I), and 774 (T→I) in respect to the FER protein from lettuce that is susceptible to Bremia (SEQ ID NO: 2 or 4) and the FER resistance protein (SEQ ID NO: 6, 8 or 10) encoded by the FER resistance gene from lettuce that shows resistance to Bremia.

### Protein Homology Modelling of the FER Resistance Protein, Characterization of Domains and Mutations

The FER resistance protein was modelled using varius software tools. Briefly, the sequence was visualized in Geneious and the protein domains were subsequently analysed with

InterPro. Protein models were created using YASARA and RoseTTAFold and SWISS-MODEL. Using NCBI's multiple alignment function a multiple alignment of the identified kinase domains were created, and subsequently transferred to Geneious. Programs PrDOS, PSIPRED, and IUPred3 were used to confirm the protein has a structure in vivo. Transmembrane prediction servers SPLIT, TMHMM, with standard settings, and PSIPRED's function MEMSAT-SVM, were used and protein property predictor RaptorX for protein domain predictions of the FER resistance protein. Disordered regions of the FER resistance protein were predicted using PrDOS with standard settings, and PSIPRED with method DISOPRED3. Mutations of the FER resistance protein in view of the resistant and susceptible plants were analysed using YASARA.

The FER resistance gene encodes for a protein that is similar to tandem protein kinases, and consists of three pseudo kinase, one kinase, and one Phloem protein 2 (PP2)-like domain. Protein homology modelling confirmed that the kinase domain has a different function to the pseudo kinase domains and that a high similarity between the three pseudo kinase domains implies these domains might have the same function. The difference between Feronia genes (from resistant and susceptible plants) that do or do not confer resistance show several mutations that differ between the resistant and susceptible plants; D678E, A704T, S708A, L756I, and T774I wherein these mutations are all present in the third domain of the FER resistance protein, which is the only real kinase domain and is proposed to bind and inhibit NLRs. Each of these mutations seem contribute to significantly change the function of the FER protein resulting in the FER resistance protein. These residues seem to function in NLR binding and inhibition. The kinase domain is proposed to get phosphorylated and ubiquitinated. Pathogens release effectors to silence this immune response, these are recognized by plant Nucleotide binding (NB) Leucine rich repeat (LRR) protein receptors (NLRs) to reinstate the hypersensitive response (HR) to kill infected cells. NLRs can be inhibited by tandem protein kinases, which are degraded upon effector recognition to activate the NLR.

1. A downy mildew resistant lettuce plant, wherein said lettuce plant comprises a FER resistance gene comprising one or more mutations, wherein the FER resistance gene encodes a protein having at least 94% sequence identity with amino acid sequence of SEQ ID NO: 2 and/or SEQ ID NO: 4 providing downy mildew resistance, wherein said FER resistance gene provides resistance to Bremia lactucae races Bl:22 and Bl:23 EU.

2. The lettuce plant according to claim 1, wherein said one or more mutations is selected from the group consisting of D678E, A704T, S708A, L756I, and T774I.

3. The lettuce plant according to claim 1, wherein the FER resistance gene comprises a coding sequence of SEQ ID NO: 5, SEQ ID NO: 7, or SEQ ID NO: 9.

4. The lettuce plant according to any one of the claim 1, wherein the FER resistance gene encodes a protein comprising amino acid sequence of SEQ ID NO: 6, SEQ ID NO: 8, or SEQ ID NO: 10.

5. The lettuce plant according to any one of the claim 1, wherein the lettuce plant is further resistant to one or more of Bremia lactucae races selected from the group consisting of races Bl:29, 31, 33, 34, 36 and 37EU.

6. The lettuce plant according to any one of the claim 1, wherein the lettuce plant is selected from the group consisting of Lactuca sativa, Lactuca virosa, Lactuca saligna, Lactuca serriola, Lactuca aculeate, Lactuca georgica, Lactuca perennis, Lactuca tatarica, and Lactuca viminea, preferably Lactuca sativa.

7. The lettuce plant according to any one of the claim 1, wherein the FER resistance gene is obtainable, derived, or originates from a lettuce plant deposited under number NCIMB 43942, NCIMB 43943, or NCIMB 43944.

8. A seed of the lettuce plant according to any one of the claim 1, comprising the FER resistance gene.

9. A FER resistance gene that confers resistance to downy mildew in lettuce plants, wherein the FER resistance gene encodes a protein that has at least 94% sequence identity with SEQ ID NO: 2 or SEQ ID NO: 4.

10. The FER resistance gene according to claim 9, wherein said FER resistance gene comprises a coding sequence of SEQ ID NO: 5, SEQ ID NO: 7, or SEQ ID NO: 9.

11. The FER resistance gene according to claim 9, wherein the FER resistance gene encodes a protein comprising amino acid sequence of SEQ ID NO: 6, SEQ ID NO: 8, or SEQ ID NO: 10.

12. The FER resistance gene according to claim 11, wherein the FER resistance gene provides resistance to at least Bremia lactucae races Bl:22 and Bl:23EU.

13. A method for identifying the downy mildew resistant lettuce plant according to claim 1, wherein the method comprises the step of establishing in the genome of a plant the presence of the FER resistance gene.

14. The method according to claim 13, wherein the step of establishing comprises establishing the presence of SEQ ID NO: 5, SEQ ID NO: 7, or SEQ ID NO: 9 in said plant.

15. Method A method for providing a lettuce plant that is resistant to downy mildew, wherein the method comprises the steps of:
a) crossing a lettuce plant comprising the FER resistance gene according to claim 9 with a lettuce plant that is susceptible to downy mildew and does not comprise said resistance gene,
b) optionally, selfing the plant obtained in step (a) for at least one time,
c) selecting plants that are resistant to downy mildew.

16. A method, comprising using a gene construct or plasmid for introducing a resistance gene into the genome of a plant or plant cell and providing resistance to downy mildew caused by one or more of B. lactucae selected from the group of races Bl:22 and Bl:23EU, wherein the gene construct or plasmid comprises the FER resistance gene according to claim 9 operably linked to expression providing sequences in said plant.

17. The FER resistance gene according to claim 12, wherein the FER resistance gene further provides resistance to Bremia lactucae races Bl:29, 31, 33, 34, 36 and 37EU.

18. A method for identifying a seed of the downy mildew resistant lettuce plant according to claim 1, wherein the method comprises the step of establishing in the genome of a seed the presence of the FER resistance gene.

19. The method according to claim 13, wherein the step of establishing comprises establishing the presence of one or more sequences selected from the group consisting of SEQ ID NO: 11 in combination with SEQ ID NO: 12, or SEQ ID NO: 13 in combination with SEQ ID NO: 14.

20. The method according to claim 16, wherein the resistance gene further provides resistance to one or more of Bremia lactucae races Bl:29, 31, 33, 34, 36 and 37EU.
